(19) United States
(12) Patent Application Publication
(10) Pub. No.: US 2021/0268122 A1
(43) Pub. Date: Sep. 2, 2021

(54) INHIBITION OF SPONTANEOUS METASTASIS VIA PROTEIN INHIBITORS OF CYSTEINE PROTEASES

(71) Applicant: DYVE BIOSCIENCES, INC., Thousand Oaks, CA (US)

(72) Inventors: Ryan Beal, Thousand Oaks, CA (US); Bruce Sand, Thousand Oaks, CA (US); Seth Brunner, Thousand Oaks, CA (US); Brandon Sand, Thousand Oaks, CA (US); Kilmar Martinez, Thousand Oaks, CA (US); Luke Gonzales, Thousand Oaks, CA (US); Lisa Misell, Thousand Oaks, CA (US); Nathan Fitzsimmons, Thousand Oaks, CA (US)

(73) Assignee: DYVE BIOSCIENCES, INC., Thousand Oaks, CA (US)

(21) Appl. No.: 17/190,329

(22) Filed: Mar. 2, 2021

Related U.S. Application Data
(63) Continuation of application No. 16/132,397, filed on Sep. 15, 2018, now abandoned.
(60) Provisional application No. 62/559,360, filed on Sep. 15, 2017.

(51) Int. Cl. A61K 47/69; A61P 35/04; A61K 45/06; A61K 38/55; A61K 9/127

(52) U.S. Cl. CPC A61K 47/6915; A61P 35/04; A61K 9/127; A61K 38/55; A61K 45/06

(57) ABSTRACT

Two disparate biological mechanisms which predispose to the dissemination and metastases of solid tumors is described. The treatment of metastatic lesions via topical and transdermal administration of therapeutic agents, such as Type 1 Cystatins, through intact skin, is directed to the inhibition of lysosomal cysteine cathepsin proteolytic enzymatic degradation of the extracellular matrix

### FIELD OF INVENTION

This invention is in the field of cancer treatment, and more particularly inhibition of spontaneous metastasis, tumor cell invasion, and lymph node colonization by means of the topical and transdermal administration of protein inhibitors of cysteine proteases.

### CROSS-REFERENCE TO RELATED APPLICATIONS

This application claims priority to U.S. application Ser. No. 16/132,397 filed Sep. 15, 2018 which claims priority to U.S. Provisional Application Ser. No. 62/559,360 filed Sep. 15, 2017 entitled ‘Inhibition of Spontaneous Metastasis via Protein Inhibitors of Cysteine Proteases’ by Bruce Sand, U.S. application Ser. No. 16/132,358 filed Sep. 14, 2018, entitled ‘Methods and Formulations For Transdermal Administration Of Buffering Agents’, and International Patent Application No. PCT/US18/51250 filed Sep. 14, 2018, entitled ‘Methods of Administration and Treatment’, all incorporated by reference in their entirety herein.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Progression to metastatic disease remains the highest mortality rate for cancer patients, despite significant efforts to therapeutically target metastatic lesions. Microenvironmental acidosis in a primary tumor increases cellular motility and invasiveness leading to increased metastasis. According to this hypothesis, acidification occurs as a result of glycolysis both in the presence of oxygen and during intermittent hypoxia, causing toxicity in the surrounding normal stroma and, thereby, providing empty space for tumor cell proliferation and invasion.

These factors lead to high concentrations of extracellular lactic acid, which may be toxic to normal and cancer cells. Many cancer cells acquire acid-resistant phenotypes that allow them to survive and proliferate when the pH is acidic.

In vivo studies have shown that solid tumors excrete acid into the surrounding parenchyma. The enhanced metastatic potential has been demonstrated to be caused by hypoxia-induced up-regulation of several metastasis-promoting matrix-degrading proteolytic enzymes, proangiogenic factors and antiapoptotic proteins. Among the proteolytic enzymes are matrix metalloproteinase-2 (MMP-2) and matrix metalloproteinase-9 (MMP-9), as well as lysosomal cysteine protease, such as cathepsin B, D or L, which may result from acid-induced lysosomal turnover and hyaluronidase with the hyaluronan receptor CD44s.

Proteases have long been considered as therapeutic targets in many disease indications involving excessive proteolysis, including cancer. Indeed, significant efforts in both the pharmaceutical industry and academia previously focused on inhibition of a major class of proteases in cancer: the matrix metalloproteinases (MMPs). The clinical failure of MMP inhibitors in the late 1990s, led to the termination of numerous drug programs.

What is needed is are new formulations and methods of administration of protease inhibitors that are effective for treating proliferative disorders associated with cancer. The inventions as described in various embodiments herein satisfies this need.

### SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

Applicants have found that the drawbacks of intravenous and oral administration of buffers and other anti-metastatic agents can be overcome by administering these agents topically and/or transdermally, but other types of administration are possible, including for example, intranasally or via transmembrane administration for example by suppository or intranasal application.

Accordingly, in one aspect a method of treating a proliferative disorder associated with cancer in a patient is provided. In some embodiments the method comprises administering an effective amount of i) one or more protease inhibitor and ii) a formulation for transdermal delivery through the skin of a subject comprising one or more buffering agent to a patient in need thereof, wherein said administration is effective to i) inhibit or prevent the metastasis of tumors or cancer cells, ii) inhibit or prevent the growth of a tumor or tumor cells, iii) inhibit or prevent carcinogenesis, iv) inhibit or prevent the intravasation of tumor cells, or v) improve or extend the duration of remission, or maintain remission of a cancer or tumor.

In one aspect, a protease inhibitor is administered transdermally. In another aspect, a protease inhibitor is co-administered with the formulation for transdermal delivery through the skin of a subject comprising one or more buffering agent. In another aspect, a protease inhibitor is formulated with the formulation for transdermal delivery through the skin of a subject comprising one or more buffering agent.

In another aspect, a protease inhibitor is administered orally, parenterally or through another route of administration that is not transdermal.

In another aspect, a protease inhibitor is administered to treat a proliferative disorder inhibits or prevents the metastasis of a tumor or cancer cells. In another aspect, a protease inhibitor is administered to prevent the growth of tumors or cancer cells. In another aspect, a protease inhibitor is administered to inhibit or prevent carcinogenesis. In another aspect, a protease inhibitor is administered to prevent the intravasation of tumor cells. In another aspect, a protease inhibitor is administered to improve or extend the duration of remission or maintains remission of a cancer or tumor.

In another aspect, a method of inhibiting or preventing metastasis of tumors is provided comprising administering an effective amount of i) one or more protease inhibitor and ii) a formulation for transdermal delivery through the skin of a subject comprising one or more buffering agent to a patient in need thereof, wherein said administration is effective to inhibit or prevent the metastasis of a tumor or cancer cells.

In another aspect, a method of improving, extending the duration of remission, or maintaining remission of a cancer or tumor is provided comprising administering an effective amount of i) one or more protease inhibitor and ii) a formulation for transdermal delivery through the skin of a subject comprising one or more buffering agent to a patient in need thereof, wherein said administration is effective to improve or extend the duration of remission or maintain remission of a cancer or tumor.

In certain embodiments, a formulation for transdermal delivery through the skin of a subject comprises a buffering agent comprising a carbonate salt in an amount between about 10-56% w/w; a penetrant portion in an amount between about 5 to 55% w/w; a detergent portion in an amount of at least 1% w/w; and wherein the formulation comprises water in an amount from 0% w/w up to 70% w/w, and wherein the formulation optionally comprises lecithin in an amount less than about 12% w/w. In other embodiments, a formulation for transdermal delivery through the skin of a subject comprises a buffering agent comprising at least one carbonate salt, lysine, tris, a phosphate buffer and/or 2-imidazole-1-yl-3-ethoxycarbonylpropionic acid (IEPA), or a combination thereof in an amount between about 10-56% w/w; and a penetrant portion in an amount between about 44 to 90% w/w, wherein the penetrant portion comprises water in an amount less than about 85% w/w, and wherein the formulation comprises less than about 12% w/w lecithin. Either of these embodiments may comprise a carbonate salt in an amount between about 7-56% w/w of the formulation.

A In another aspect, a chemotherapeutic or immunotherapeutic agent is co-administered with one or more protease inhibitor and/or one or more buffering formulation provided herein.

### BRIEF DESCRIPTIONS OF THE DRAWINGS

The following invention will be better understood with reference to the specification, appended claims, and accompanying drawings, where:

FIG. 1 illustrates the two-compartment “bricks and mortar” model of the stratum corneum (SC) and the pore pathway within the SC pathway.

FIG. 2 shows the pathways into the skin for transdermal drug delivery of skin agents. A. transdermal transport via within extracellular lipids. B. transport through hair follicles and sweat ducts. C. transport directly across the SC. D. stripping, ablation and microneedles produce larger pathways across the SC.

FIG. 3 depicts the hydrophilic and lipophilic pathways for drug penetration and action mode of penetration enhancers.

FIG. 4 illustrates the comparison of the three dimensional structures of stefin A and cystatin C.

FIG. 5 illustrates the mode of determining micellar stability.

FIG. 6 is a schematic illustration of polymer micelle formation

FIG. 7 is a schematic of the reverse micellar structures formed by lecithin with and without bile salt.

FIG. 8 shows the effect of adding electrolytes to the bile salt/lecithin micelles resulting in increase in viscosity and stability.

FIG. 9 represents a photomicrograph of the treated site with greater abundance of collagen and characteristics that depict a more recently deposited fibrous network. The epidermal layer is much thicker, well organized and reflects a greater cellular metabolic activity.

FIG. 10 represents a photomicrograph of the untreated control site.

FIG. 11 illustrates the TEWL measurements of the right and left arms of subjects showing increased transdermal water loss following topical applications of the chemical permeation enhancement formulations.

FIG. 12 illustrates the TEWL measurements of the right and left arms of subjects showing increased transdermal water loss following topical applications of the chemical permeation enhancement formulations.

FIG. 13 is an amplification plot data using pro-collagen primers and probes. These results show that human dermal fibroblast cells began expressing pro-collagen within 30 minutes after exposure to sample. Control samples exposed to base alone did not express pro-collagen at this time point.

FIG. 14 documents the most frequently observed permeation enhancement formulations with regard to enhancement ratios and synergism as revealed from electrometric studies of skin conductance.

FIG. 15 depicts the concentration mass of iron (Fe) in samples collected at four different time points. Samples were evaluated by PIXI analysis. Donor sample at the concentration used had Fe at a concentration mass of 169.708 (straight line). Experimental samples started showing an increase in the concentration mass of Fe at 30 minutes and reached a peak value in 120 minutes. Fe was undetectable in well incubated with base of PBS.

FIG. 16 (Table 1) depicts the concentration mass of copper (Cu) in samples collected at four different time points. Samples were evaluated by PIXI analysis. Donor sample at the concentration used had Cu at a concentration mass of 3.132 (straight line). Experimental samples started showing an increase in the concentration mass of Cu at minutes and reached a peak value in 120 minutes. Cu was undetectable in well incubated with base of PBS. Documents the characteristics of the various members of the cystatin super-family in humans.

### DETAILED DESCRIPTION

The practices described herein employ, unless otherwise indicated, conventional techniques of tissue culture, immunology, molecular biology, microbiology, cell biology and recombinant DNA, which are within the skill of the art. See, e.g., Harlow and Lane eds. (1999) Antibodies, A Laboratory Manual and Herzenberg et al. eds (1996) Weir's Handbook of Experimental Immunology.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof. On the other hand “one” designates the singular.

As used herein, the term “comprising” is intended to mean that the compositions and methods include the listed elements, but do not exclude other unlisted elements. “Consisting essentially of” when used to define compositions and methods, excludes other elements that alters the basic nature of the composition and/or method, but does not exclude other unlisted elements. Thus, a composition consisting essentially of the elements as defined herein would not exclude trace amounts of elements, such as contaminants from any isolation and purification methods or pharmaceutically acceptable carriers, such as phosphate buffered saline, preservatives, and the like, but would exclude additional unspecified amino acids. “Consisting of” excludes more than trace elements of other ingredients and substantial method steps for administering the compositions described herein. Embodiments defined by each of these transition terms are within the scope of this disclosure and the inventions embodied therein.

As noted above, one aspect of the invention is a method to inhibit cancer growth and metastasis, including diminution of cancer mass by non-systemic parenteral, including topical administration of antimetastatic agents, including those agents that result in buffering the immediate environment of tumor cells, including solid tumors and melanomas. For non-systemic parenteral administration, such as intramuscular, intraperitoneal or subcutaneous administration standard formulations are sufficient. These formulations include standard excipients and other ancillary ingredients such as antioxidants, suitable salt concentrations and the like. Such formulations can be found, for example, in Remington's Pharmaceutical Sciences (13^{th} Ed), Mack Publishing Company, Easton, Pa.—a standard reference for various types of administration. As used herein, the term “formulation(s)” means a combination of at least one active ingredient with one or more other ingredient, also commonly referred to as excipients, which may be independently active or inactive. The term “formulation”, may or may not refer to a pharmaceutically acceptable composition for administration to humans or animals, and may include compositions that are useful intermediates for storage or research purposes. In an embodiment, administration to humans or animals may include, without limitation, topical, sublingual, rectal, vaginal, transdermal, trancutaneous, oral, inhaled, intranasal, pulmonary, subcutaneous, pulmonary, intravenous, enteral or parenteral. Suitable topical formulations for transdermal administration of active agents for the methods provided herein are described in U.S. Ser. No. 14/757,703, to Sand B., et al., incorporated herein by reference in it's entirety. Suitable penetrants are described, for example, in PCT publications WO/2016/105499 and WO/2017/127834.

As the patients and subjects of the invention method are, in addition to humans, veterinary subjects, formulations suitable for these subjects are also appropriate. Such subjects include livestock and pets as well as sports animals such as horses, greyhounds, and the like.

In an embodiment, a “pharmaceutical composition” is intended to include, without limitation, the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” refers, without limitation, to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will, without limitation, vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effected may, without necessarily being therapeutic, also be a cosmetic effect, in particular for treatment for disorders of the skin described herein.

In an embodiment, a “subject” of diagnosis or treatment is, without limitation, a prokaryotic or a eukaryotic cell, a tissue culture, a tissue or an animal, e.g. a mammal, including a human. Non-human animals subject to diagnosis or treatment include, for example, without limitation, a simian, a murine, a canine, a leporid, such as a rabbit, livestock, sport animals, and pets.

In an embodiment, as used herein, the terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

### Proteases and Inhibitors Thereof

Cysteine cathepsin are synthesized as inactive precursors, which are normally activated in the acidic environment of lysosomes, where they function primarily as intracellular proteases that mediate terminal bulk proteolysis. Furthermore, in some of these cancers, the changes in cysteine cathepsin expression or activity have diagnostic or prognostic value. In term of which cysteine cathepsins are specifically involved in cancer, cysteine cathepsin B and L have been investigated most intensively.

In normal cells, cysteine cathepsins are usually located in lysosome compartments in the cellular plasma membrane, whereas, during cancer progression they move to the cell surface, from where they can be secreted into the extracellular milieu. Furthermore, because the extracellular microenvironment of tumors is acidic, cysteine cathepsin proteases can still function outside the lysosome. This change in cellular localization has important implications for the therapeutic efficacy of cysteine cathepsin inhibitors, because small-molecule inhibitors that do not enter cells could have a potent effect by targeting cell surface or secreted cysteine cathepsins, but would leave the intracellular cysteine cathepsins in normal cells untouched, thereby minimizing toxicity.

An assay of cathepsin B in metastatic cancer has revealed that the activity of this protease secreted into the media was increased up to 4-fold. Thus, it appears that the acid pH of tumors can induce the release of cathepsin involved in extracellular matrix (ECM) turnover.

Metastasis in, at least, two cell lines; MDB-MB-231 (human breast adenocarcinoma) and PC-3M (prostate adenocarcinoma) are effectively inhibited by buffer therapy to neutralize the acidic microenvironment. The applicant was, however, surprised to learn that B16-F10 (murine melanoma) and LL/2 cells (murine lung carcinoma) were resistant to the same therapeutic agents. These findings have led to the realization that since buffering is not universally efficacious, resistant and sensitive lines might utilize different metastatic mechanisms; one that is pH-independent and one that is pH-dependent. Metabolic profiling confirms that buffering-resistant cells have much more actively expressed proteases in a pH-independent fashion, compared to sensitive lines whose protease activities are lower and pH-dependent. Acidic pH, results in morphological changes in sensitive cells, while resistant cells remain unaffected.

It appears that sensitive cells activate proteases and alter their morphology by acidifying their microenvironment, which can be inhibited by buffer therapy. Resistant cells, however, have constitutively active protease release, by means of constitutive secretion, proteins are secreted from a cell continuously, regardless of external factors or signals. It has been shown that resistant cells are also significantly smaller with a less energetic glycolytic phenotype both of which may allow for more rapid extravasation during metastasis.

Based upon this revelation, an in-depth study of the lysosomal cysteine proteases, such as cathepsin B, D or L, and their relation to metastasis of solid tumors, has been undertaken. Cysteine cathepsins are optimally active in a slightly acidic pH and are mostly unstable at neutral pH. Cathepsins with strong elastolytic and collagenolytic activities are known to be chiefly responsible for the remodeling of the extracellular matrix (ECM), which predisposes to cancer metastasis.

It is now clear that the cathepsins have an important role in both tumor progression and invasion. Cathepsin B specifically was first linked to cancer some 30 years ago and has been shown to be associated with cancer progression and/or activity in several different types of tumors. Moreover, the level of cathepsin expression positively correlated with a poor prognosis for cancer patients. As such, the cathepsins B, L and others have been shown to promote the migration and invasion of tumor cells. In addition to their well-known function associated with the ECM degradation and remodeling in the tumor microenvironment, cathepsins have been revealed to participate in the proteolytic cascade activation.

Another level of complexity is introduced by the fact that the proteolytic activity of cysteine cathepsins is regulated by their endogenous protein inhibitors, stefins, cystatins and serpins. It has been found that a higher level of cathepsin inhibitors in different types of cancer correlate with a favorable prognosis for cancer patients. This revelation has laid the foundation for this invention.

The cancer state is currently viewed as a product of its microenvironment. Therefore, new technologies that enable the targeting of the tumor microenvironment would represent an efficient approach to cancer prevention and intervention. Indeed, such a technology was recently provided by the development of a novel, directed, drug-delivery system enabling the targeting of the tumors, their microenvironment, and the matrix degrading cysteine cathepsin proteases.

The inventor's realization that proteolysis is necessary for several stages in the development of invasive and metastatic cancers emphasizes the therapeutic importance of unequivocally identifying the key tumor-promoting proteases and developing successful strategies to inhibit their functioning.

The major regulators of the mature cysteine cathepsins are their endogenous protein inhibitors, cystatins, thyropins and serpins. Based upon their physiologic role, they are divided into emergency and regulatory inhibitors. Typical emergency inhibitors are cystatins, which are separated from their target enzymes and primarily act on escaped proteases or proteases of invading pathogens.

The respective anti-tumor therapy using JPM-565 in other cases has, however, proved to be very successful, thereby resulting in a significant reduction in tumor growth. This has proved the concept of stroma targeting with cathepsins as potent targets in cancer treatment.

Several approaches have been developed to block cysteine cathepsin activity, including small-molecule inhibitors, antibodies and increased production of endogenous inhibitors (the cystatins and stefins). As most of the targeted agents that have been developed are small-molecules inhibitors (average molecular mass: 350 Da), the focus is on those used successfully in preclinical and clinical studies and that have shown efficacy in vivo and thus demonstrate the most promise for therapeutic applications.

The development of cysteine cathepsin inhibitors has followed the traditional process used for protease inhibitors: that is large libraries of natural products or synthetic compounds followed by smaller focused screens. The main classes of cysteine cathepsin inhibitors are nitriles, vinyl sulfones and epoxysuccinyl-based compounds, which are either broad-spectrum or selective for individual family members. All of these inhibitors are directed to the active site and depending on their mechanism of action, can be further classified into covalent or non-covalent binders and reversible or irreversible inhibitors.

Cellular proteases are regulated at many levels, one being the interaction with endogenous inhibitors. In 1957, the first heat stable inhibitor of cysteine cathepsin B was described. Cysteine protease inhibitors (CPIs) are very tight binding, pseudoirreversible inhibitors. Endogenous CPIs constitute a single protein superfamily, the cystatins, such as type 1 cystatins; stefins A (or cystatins A) with a molecular mass of 11,775 Da and stefins B (or cystatins B) with a molecular weight of 11,006 Da. These agents are bioavailable and counter balancing inhibitors of the over-expressed tumor-associated cysteine cathepsin proteolytic activity.

Driven by the lack of buffer-induced inhibition of metastasis in cellular resistant tumors and the poor bioavailability in the administration of specific endogenous protease inhibitors in selective tumors, this invention was conceived to enable the topical and transdermal bioavailable administration of all effective cysteine cathepsin protease inhibitors.

In another aspects, embodiments of the invention provided herein include a topical and transdermal cysteine cathepsin protease inhibitor delivery system, which will intercede in the matrix degradation process enabled by the up-regulation of cysteine cathepsin protease irrespective of the pH-independent cell-resistant mechanism enabling the up-regulation of matrix-degrading enzymes.

Invasive cancer develops from solid tumors cycling through multiple stages of somatic evolution. Heritable changes are driven by the hostile microenvironment. Low extracellular pH with acidity is a major hallmark of the hostile tumor microenvironment and a driver of metastatic potential in solid tumors, such as breast, hepatic and prostate.

The extracellular pH of malignant solid tumors is acidic, in the range of 6.5 to 6.9, whereas the pH of normal tissues is significantly more alkaline, 7.2 to 7.5. These observations have led to the “acid-mediated invasion hypothesis,” wherein tumor-derived acid facilitates tumor invasion by promoting normal cell death and extracellular matrix (ECM) degradation of the parenchyma surrounding growing tumors.

The transdermal delivery of sodium bicarbonate, or other buffering agents, satisfies the hypothesis that inhibition of tumor metastasis is due to increased “buffering” of interstitial fluid of either the primary or the metastatic tumors and circumvents the poor bio-availability associated with first-pass metabolism, as well as the common gastro-intestinal side effects of oral dosing.

However, this effect is not universal as was previously observed. This applicant was surprised to learn that metastasis is not inhibited by buffers in some tumor models, regardless of buffering agent implemented. It has been revealed that B16-F10 (murine melanoma), LL/2 (murine lung) and HCT116 (human colon) tumors are resistant to treatment with lysine buffer therapy, whereas, metastasis is potentially inhibited by lysine buffers in MDA-MB-231 (human breast) and PC3M (human prostate) tumors. Work by others have confirmed that sensitive cells utilize a pH-dependent mechanism for successful metastasis supported by a highly glycolytic phenotype that acidifies the local tumor microenvironment resulting in morphologic changes. In contrast, buffer-resistant cell lines exhibit a pH-independent metastatic mechanism involving constitutive secretion of matrix-degrading proteases without elevated glycolysis. By means of constitutive secretion, proteins are secreted from a cell continuously, regardless of external factors or signals. These revelations have identified two distinct mechanisms of experimental metastasis, one of which is pH-dependent (buffer therapy sensitive cells) and one which is pH-independent (buffer therapy resistant cells).

In addition to faster growth rates in vivo, resistant cells are significantly smaller in diameter than sensitive cells, which may allow increased access to invade the extracellular space, either through more efficient extravasation or secondary site colonization. Faster growth and smaller size may be enough to render resistant cells too aggressive for buffer therapy to be effective.

Other important molecular and metabolic parameters may contribute to resistance. Sensitive cells, for example, are unequivocally more glycolytic that resistant cells. Cells with elevated glycolysis produce more acidic tumor microenvironment.

To determine glycolytic activity, a “glycolytic stress test” has been performed, which includes the measuring of extracellular acidification rates (ECAR) after sequential addition of glucose to measure basal glycolysis, a mitochondrial poison, (oligomycin) to estimate total glycolytic capacity, and 2-deoxyglucose to measure non-glycolytic ECAR. Interestingly, sensitive cells have a significantly higher basal glycolytic rates, compared to resistant cells. Glycolytic reserve is calculated by measuring the difference in the maximal glycolytic capacity, after treatment with oligomycin, and basal glycolysis. Possibly, as a consequence of their high basal rates, the sensitive cells showed significantly lower amounts of glycolytic reserve, compared to resistant cells, suggesting that they are near maximum glycolytic capacity in their basal metabolic state.

Invasion kinetics and metabolic profiling suggest the resistant cell invade via a mechanism that is pH-independent. Proteases have been identified as key enzymes involved in the metastatic cascade. Prior data have shown that low pH significantly stimulated the releases of active cathepsin-B from buffer-sensitive MDA-MB-231 cells in culture. Hence, it appears that resistant cells may release active proteases in a constitutive, pH-independent fashion.

Among the hydrolytic machinery thought at one time to reside in the lysosomal compartment of the cellular plasma membrane, are proteases among which are the cysteine cathepsins, members of the family of papain-like cysteine proteases. The view of cysteine cathepsins as lysosomal proteases is, however, changing as there is now clear evidence of cathepsin localization in other cellular compartments, as well. Together with the growing number of non-endosomal roles is their involvement in diseases, such as cancer.

Expression, release, and enzymatic activity of proteases are primarily regulated by acidosis. While sensitive cells have measurable expression of MMPs, resistant cells have consistently higher expression of MMPs regardless of pH. Expression of MMPs correlate with proteases activity in vivo. Also acidic pH has been shown to increase pericellular active cysteine cathepsins in vitro. In addition, serum cathepsin levels have been positively correlated with metastasis. The roles of cathepsins in the individual tumor-biological processes were also confirmed offering new possibilities for the diagnosis and treatment of cancer.

In cancer, the inhibitory effect of cystatins and other small molecule cysteine cathepsin protease-inhibitors could beneficially counteract metastasis-associated proteolytic activity, which is enabled by ECM-degradation. These so-called resistant cell lines enhance ECM degradation, while constitutive secretion of cysteine cathepsin is involved in a number of normal and pathological conditions, such as immunomodulatory functions, by controlling the activity of cysteine proteases or by other mechanisms not related to their inhibitory functions.

Formulations and therapeutic compositions provided herein are used in methods of treating many cancers, including but not limited to breast cancer, prostate cancer, pancreatic cancer, lung cancer, bladder cancer, skin cancer, colorectal cancer, kidney cancer, hepatic cancer, and thyroid cancer.

Formulations and therapeutic compositions provided herein are also used in methods of treating a cancer or tumor, including but not limited to adrenocortical carcinoma, basal cell carcinoma, bladder cancer, bone cancer, brain tumor, breast cancer, cervical cancer, colon cancer, colorectal cancer, esophageal cancer, retinoblastoma, gastric (stomach) cancer, gastrointestinal tumors, glioma, head and neck cancer, hepatocellular (liver) cancer, islet cell tumors (endocrine pancreas), kidney (renal cell) cancer, laryngeal cancer, non-small cell lung cancer, small cell lung cancer, medulloblastoma, melanoma, pancreatic cancer, prostate cancer, renal cancer, rectal cancer, and thyroid cancer.

While preferred embodiments of the methods provided herein are typically directed to a particular cancer, solid tumor or grouping thereof, a more complete but still non-limiting listing of suitable cancers and tumors that may be tested for effectiveness according to embodiments provided herein includes the following: lymphoblastic leukemia (ALL), acute myeloid leukemia (AML), adrenocortical carcinoma, aids-related cancers, kaposi sarcoma (soft tissue sarcoma), aids-related lymphoma (lymphoma), primary cns lymphoma (lymphoma), anal cancer, astrocytomas, atypical teratoid/rhabdoid tumor, childhood, central nervous system (brain cancer), basal cell carcinoma, bile duct cancer, bladder cancer. childhood bladder cancer, bone cancer (includes ewing sarcoma and osteosarcoma and malignant fibrous histiocytoma), brain tumors, breast cancer, childhood breast cancer, bronchial tumors, burkitt lymphoma (non-hodgkin lymphoma, carcinoid tumor (gastrointestinal), childhood carcinoid tumors, cardiac (heart) tumors, central nervous system tumors. atypical teratoid/rhabdoid tumor, childhood (brain cancer), embryonal tumors, childhood (brain cancer), germ cell tumor (childhood brain cancer), primary cns lymphoma, cervical cancer, childhood cervical cancer, cholangiocarcinoma, chordoma (childhood), chronic lymphocytic leukemia (CLL), chronic myelogenous leukemia (CML), chronic myeloproliferative neoplasms, colorectal cancer, childhood colorectal cancer, craniopharyngioma (childhood brain cancer), cutaneous t-cell lymphoma, ductal carcinoma in situ (DCIS), embryonal tumors, (childhood brain CNS cancers), endometrial cancer (uterine cancer), ependymoma, esophageal cancer, childhood esophageal cancer, esthesioneuroblastoma (head and neck cancer), Ewing sarcoma (bone cancer), extracranial germ cell tumors, extragonadal germ cell tumors, eye cancer, childhood intraocular melanoma, intraocular melanoma, retinoblastoma, fallopian tube cancer, fibrous histiocytoma of bone (malignant, and osteosarcoma), gallbladder cancer, gastric (stomach) cancer, childhood gastric (stomach) cancer, gastrointestinal carcinoid tumor, gastrointestinal stromal tumors (gist) (soft tissue sarcoma), childhood gastrointestinal stromal tumors, germ cell tumors, childhood central nervous system germ cell tumors, childhood extracranial germ cell tumors, extragonadal germ cell tumors, ovarian germ cell tumors, testicular cancer, gestational trophoblastic disease, hairy cell leukemia, head and neck cancer, heart tumors, hepatocellular (liver) cancer, histiocytosis (Langerhans cell cancer), Hodgkin lymphoma, hypopharyngeal cancer (head and neck cancer), intraocular melanoma, childhood intraocular melanoma, islet cell tumors, (pancreatic neuroendocrine tumors), Kaposi sarcoma (soft tissue sarcoma), kidney (renal cell) cancer, Langerhans cell histiocytosis, laryngeal cancer (head and neck cancer), leukemia, lip and oral cavity cancer (head and neck cancer), liver cancer, lung cancer (non-small cell and small cell), childhood lung cancer, lymphoma, male breast cancer, malignant fibrous histiocytoma of bone and osteosarcoma, melanoma, childhood melanoma, melanoma (intraocular eye), childhood intraocular melanoma, Merkel cell carcinoma (skin cancer), mesothelioma, childhood mesothelioma, metastatic cancer, metastatic squamous neck cancer with occult primary (head and neck cancer), midline tract carcinoma with nut gene changes, mouth cancer (head and neck cancer), multiple endocrine neoplasia syndromes—see unusual cancers of childhood, multiple myeloma/plasma cell neoplasms, mycosis fungoides (lymphoma), myelodysplastic syndromes, myelodysplastic/myeloproliferative neoplasms, myelogenous leukemia, chronic (CML), myeloid leukemia, (acute AML), myeloproliferative neoplasms, nasal cavity and paranasal sinus cancer (head and neck cancer), nasopharyngeal cancer (head and neck cancer), neuroblastoma, non-hodgkin lymphoma, non-small cell lung cancer, oral cancer (lip and oral cavity cancer and oropharyngeal cancer), osteosarcoma and malignant fibrous histiocytoma of bone, ovarian cancer, childhood ovarian cancer, pancreatic cancer, childhood pancreatic cancer, pancreatic neuroendocrine tumors (islet cell tumors), papillomatosis, paraganglioma, childhood paraganglioma, paranasal sinus and nasal cavity cancer, parathyroid cancer, penile cancer, pharyngeal cancer, pheochromocytoma, childhood pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, recurrent cancer, renal cell (kidney) cancer, retinoblastoma, rhabdomyosarcoma, salivary gland cancer, sarcoma, childhood rhabdomyosarcoma (soft tissue sarcoma), childhood vascular tumors (soft tissue sarcoma), Ewing sarcoma (bone cancer), Kaposi sarcoma (soft tissue sarcoma), osteosarcoma (bone cancer), soft tissue sarcoma, uterine sarcoma, Sézary syndrome (lymphoma), skin cancer, childhood skin cancer, small cell lung cancer, small intestine cancer, soft tissue sarcoma, squamous cell carcinoma of the skin, squamous neck cancer with occult primary, stomach (gastric) cancer, childhood stomach, t-cell lymphoma, testicular cancer, childhood testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter kidney (renal cell cancer), ureter and renal pelvis (transitional cell cancer kidney renal cell cancer), urethral cancer, uterine cancer (endometrial), uterine sarcoma, vaginal cancer, childhood vaginal cancer, vascular tumors (soft tissue sarcoma), vulvar cancer, and Wilms tumor (and other childhood kidney tumors).

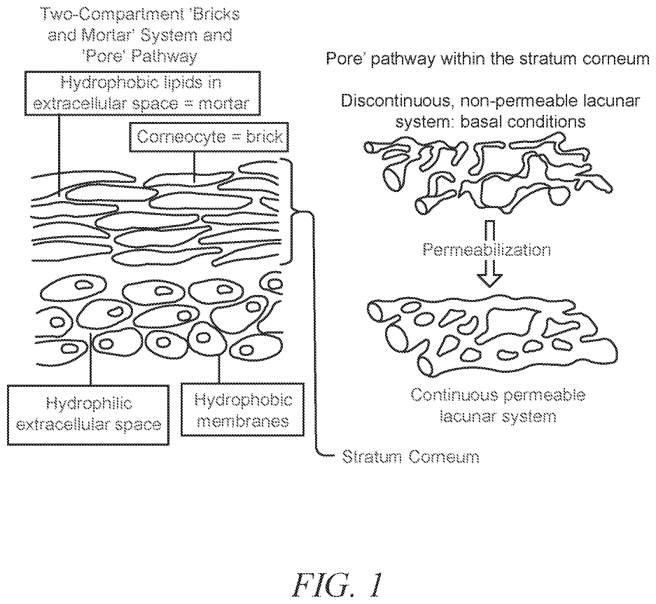

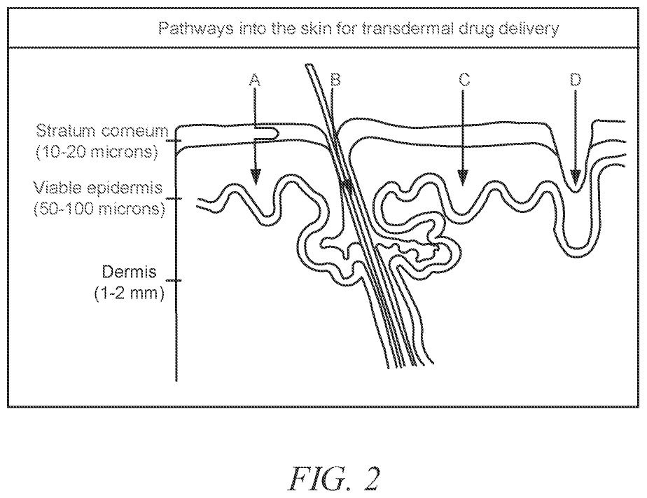

Incorporated in this patent is a topical and transdermal drug delivery technology facilitating the effective and expeditious delivery and bioavailability of cystatins and other small molecular cysteine cathepsin protease-inhibitors in therapeutic dosimetry. Skin, however, presents a formidable permeation barrier to most topically applied active agents. This barrier resides in the superficial layer of the epidermis, the stratum corneum (SC) and has been compared to a two-compartment “bricks and mortar” model (FIG. 1). Various pathways are available for transepidermal drug delivery (FIG. 2).

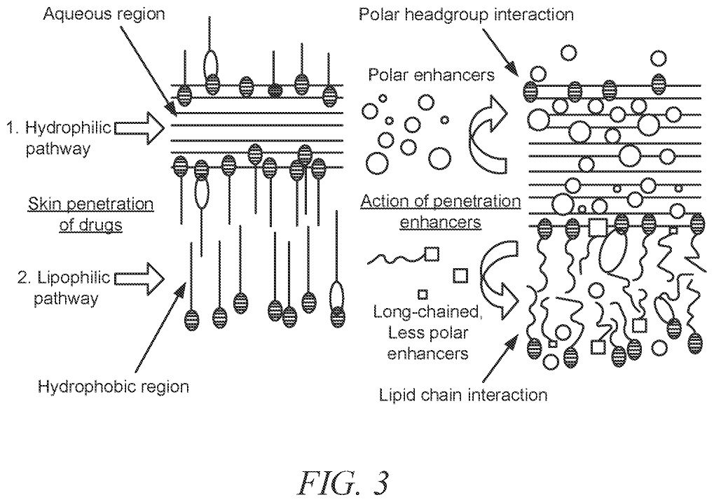

The applicable hydrophilic and lipophilic pathways for drug penetration and their action modes are illustrated by FIG. 3. This patent embodies technology, which breaches the barriers presented by the SC.

The following detailed description is of the best currently contemplated modes for carrying out the invention. The description is not to be taken in a limited sense, but is merely for the purpose of illustrating the general principles of the invention, since the scope of the invention is best defined by the appended claims.

Microenvironmental acidosis in a primary tumor increases cellular motility and invasiveness leading to increased metastasis. During primary tumor development, cell metabolism is often altered resulting in up-regulated glycolysis and acidosis leading to tumor metastasis.

In vivo studies have shown that solid tumors export acid into the surrounding parenchyma. The enhanced metastatic potential has been demonstrated to be caused by hypoxia-induced up-regulation of several metastasis-promoting matrix-degrading proteolytic enzymes, proangiogenic factors and antiapoptotic proteins. Among the proteolytic enzymes are matrix metalloproteinase-2 (MMP-2) and matrix metalloproteinase-9 (MMP-9), as well as lysosomal cysteine protease, such as cathepsin B, D or L, which may result from acid-induced lysosomal turnover and hyaluronidase with the hyaluronan receptor CD44s.

An assay of cathepsin B in metastatic cancer has revealed that the activity of this protease secreted into the media was increased up to 4-fold. Thus, it appears that the acid pH of tumors can induce the release of cysteine cathepsin protease involved in extracellular matrix (ECM) turnover.

Recent data suggest proteases of the papain-like cysteine cathepsin family as molecular targets for cancer therapy. Proteases contribute to invasion and metastasis of solid tumors by degradation of extracellular matrix proteins and by shredding of bioactive peptides. Elevated expression and/or activity of certain endosomal/lysosomal cysteine proteases, i.e., cysteine cathepsins of the papain protease family, correlate with increased malignancy and poor prognosis for patients.

Addressing the microenvironmental acidosis by means of buffering has demonstrated an inhibition of metastasis and colonization in the mouse model. Recent studies have revealed a reduction of acidity through systemic buffers significantly inhibiting development and growth of metastases in mouse xenograft.

The applicant has recently become aware of studies, which appear to confirm that certain cell-lines are resistant to the functionality of buffering of the tumor microenvironment. In other words, the effect of buffering is “not universal.” That is, there are some tumors, which manifest a pH-independent metastasis involving a constitutive secretion of cysteine cathepsin matrix-degrading proteolytic enzymes and other cell-lines that demonstrate a pH-dependent mechanism supported by a highly glycolytic phenotype and acidosis that drives a similar morphological matrix-destructive process predisposing to spontaneous tumor cell invasion and metastasis.

These two processes each up-regulate matrix destructive proteolytic enzymes but are driven by different mechanisms. This patent supports the hypothesis that effectively inhibiting the production of lysosomal cysteine cathepsin proteolytic enzymes will serve to inhibit spontaneous metastasis of solid tumors, irrespective of the metastatic mechanism.

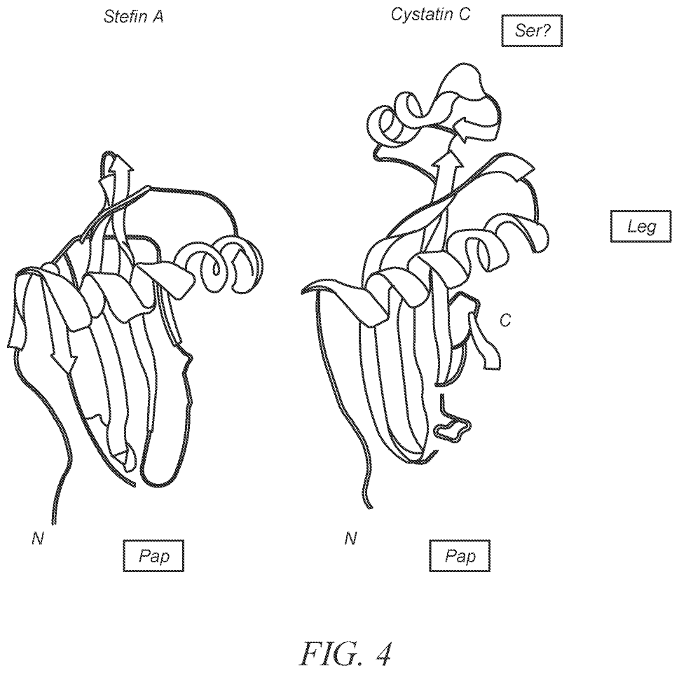

Lysosomal cysteine cathepsin proteolytic enzymes appear to be constitutively secreted into the ECM associated with pH-independent solid tumor cell-lines. These enzymes are responsible for the matrix degradation, which predispose to the spread, metastasis and colonization of these solid tumors. This patent embodies effective and expeditious delivery of cysteine cathepsin protease-inhibitors including, but not limited by, Type I Cystatins, also named cystatin A or Stefins A and Cystatin B or Stefins B of molecular mass, 11,175 Da and 11,006 Da, respectively (FIG. 4). Further employed in this patent are cysteine cathepsin protease-inhibitors, but not limited by, small-molecule inhibitors; such as epoxysuccinyl-based inhibitor E-64 (L-trans-Epoxysuccinyl-leucylamido (4-guanidino) butane), a thiol protease inhibitor, of which JPM-565 is a derivative and which has been very potent in the treatment of pancreatic islet tumors in a mouse model, as well as the cell-permeable broad spectrum cysteine cathepsin protease-inhibitor; JPM-OEt, active against early and advanced mammary cancer stages in the MMTV-PyMT-transgenic mouse model. Each of these therapeutic agents are to be administered in appropriate therapeutic doses up to 100 mg/kg per day (Table 1.)

The essence of this patent is the inhibition of metastasis of certain cancer cell-lines, which is independent of the functionality of pH buffering. In other words, this patent embodies a mechanism in which the matrix-degradation driven by the up-regulation of cysteine cathepsin proteases is inhibited. Recent studies have revealed that there are some tumors, which manifest a pH-independent metastasis involving a constitutive secretion of cysteine cathepsin matrix-degrading proteolytic enzymes, while other cell lines may demonstrate a significant pH-dependent mechanism supported by a highly glycolytic phenotype and acidosis. This mechanism drives a similar morphological matrix-destructive process, which drives spontaneous tumor cell invasion and metastasis.

This invention supports the topical and transdermal delivery of members of the family of cystatin-based inhibitors of cysteine cathepsin matrix-degrading proteolytic enzymes in the absence of buffering activity of the acidic tumor microenvironment. These therapeutic agents are conveyed to the site of the potential cysteine cathepsin-driven matrix degradation.

The molecular masses of each cystatin-based cysteine cathepsin places a burden on the drug delivery system because the therapeutic agent is in the category of a macromolecule. The inventive delivery system has been specifically designed to host these large molecules as described in the following disclosure. Measurement of cysteine cathepsin activities after intra-peritoneal injections of JPM-OEt revealed effective inhibition of the protease in pancreas, kidney, and liver while activities in mammary and in lungs were not significantly affected due to the pharmacokinetic properties, which resulted in poor bioavailability.

The topical and transdermal drug delivery technology of this invention employs, bioavailability without limitation, two different vector technologies, nano-scaled chemical permeation enhancement formulations (CPEs) and cell penetrating peptides (CPPs), which might be used separately or in synergistic combination, dependent upon the agent to be delivered.

In certain embodiments, alternative methods of administering agents or drugs through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include, without limitation, microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ionto system (DEP), TTS transdermal therapeutic systems, membrane-moderated systems (drug reservoir totally encapsulated in a shallow compartment), adhesive diffusion-controlled system (drug reservoir in a compartment fabricated from drug-impermeable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

The application of CPPs in combination with CPEs to enhance the transdermal drug delivery (TDDD) is particularly intriguing because the SC presents a formidable barrier to the penetration of peptides, especially of a molecular weight above 500 Da. The ability of peptides to, thereby function as penetration enhancers was not only unexpected but, indeed, counterintuitive.

Also embodied in this patent is the topical administration of agents and drugs, with or without occlusion in any manner and which are not conjugated with or delivered by means of penetration enhancing formulations, but are merely applied to the intact skin with or without massaging the skin for the purpose of breaching the skin's permeation barrier.

The applicant surprisingly discovered that the combination of these two hypothetical mechanisms, functioning in synergy, was successful in TDDD of guest molecules of molecular weights exceeding 500 Da and, in fact, beyond 150 kDa. These two synergistic mechanisms involve different interactions between the SPPs and the cellular moiety in the “transcellular” mechanism and the CPEs in the “extracellular” mechanism.

This invention discloses integrative and cooperative methods with compositions that are directed to the simultaneous and selective disruption of the cellular and lipid matrix contributions to the SC permeation barrier in conjunction with the transdermal delivery of agents. The mode of each physico-chemical component will be presented separately, although they may participate cooperatively in a chemical permeation enhancement (CPE) composition.

While primarily directed to the permeation enhancement of drug delivery for human beings, the application of this invention is not limited to humans, but has similar application to other members of the animal kingdom.

While it has been well recognized that the primary efforts employed to enhance SC permeability have focused upon manipulations of the extracellular lipid milieu, little attention has been directed to degrading the cellular components of the SC. This patent embodies an integrative and cooperative transdermal drug delivery formulation that simultaneously disrupts both the extracellular lipid matrix, as well as the cellular contribution to the SC permeation barrier. This patent embodies the application of permeation enhancement formulations directed either to the extracellular lipid matrix, the transcellular structure or both in co-administration. This is to be determined by the nature of the guest cargo, its application, its target site and its molecular weight.

The preferred biochemical process, which is directed to the cellular component of the SC permeability barrier, is facilitated by a synergistic action of several biological processes, which combine to enhance transdermal drug delivery. Each of these processes might be used individually.

This patent embodies TD-1, as well as the other cationic cyclo-peptide variants identified as TDR-2, TDR-3 and TDR-7, in which arginine substitutions are made at N-4, N-5 and N-7, and TDK-2, TDK-3 and TDK-7, in which lysine substitutions are made at N-2, N-3 and N-7. Also embodied in this patent is cationic cyclo-peptide variant TD-34 as bis-substitute peptide in N-5 and N-6. The cyclic structure and the disulfide constrained nature is critical for enhancement activity of the peptides. The TDS series of the same amino acid sequence of cyclic structure with TD-1 is further embodied as a modification via substitution of the N-terminal with three amino acids possessing the same cationic group with various side-chain lengths. The enhancement activity has been demonstrated to be proportional to side-chain length and identified as TDS-3>TDS-2>TDS-1.

While the exact mechanism is unclear, our studies have revealed the profound activity of cell penetrating peptides (CPPs) with special reference to TD-1, to be upon interactions with the skin cellular components. The CPPs function by permeating through the transcellular route passing through hydrophilic keratin-packed corneocytes that are embedded in multiple hydrophobic lipid bilayers. While partitioning into the keratin-rich corneocytes, they form bridges that bind with the filamentous keratin α-helices via hydrogen bonds in co-administration as peptide-chaperones without interacting with the guest cargo or degrading the lipid matrix. SPPs, in fact, enhance the lipid organization while simultaneously increasing skin electrical conductivity. TD-1 is non-cytotoxic and non-irritating to skin.

It has been demonstrated that the CPPs also utilize the intercellular pathways via small gaps between the corneocytes by disrupting cell-to-cell junctional desmosomes expeditiously, thereby modifying the normal ultrastructural spacing from about 30 nm to about 466 nm in as little as 30 minutes from topical administration. Transmission electron microscopy has revealed that the intercellular gaps are a transient process that will escort macromolecules across the SC permeation barrier restoring the breaches in about one hour after application.

The co-administration of CPPs has been postulated to result in a statistically significant increase in percentage of α-helices of keratins, suggesting that CPPs stabilize these structural proteins (keratins). The intra-cellular keratins are stabilized by disulfide bonds, which are tightly packed either in α-chains (α-keratins) or in β-sheet (β-keratins) structures. The high-degree of cross-linking by the disulfide bonds, hydrophobic interactions and hydrogen bonds between the keratin filament structures within the individual corneocytes confer its mechanical stability preventing free drug transport.

Keratolytic agents will disrupt the tertiary structure and hydrogen bonds between individual keratin filaments, thereby promoting penetration through intact skin. The administration of keratolytic agents will release keratin-bound active drug and enhance bioavailability.

One biochemical process is deployed to disrupt the disulfide linkage of the keratin filaments of which the corneocytes of the SC are comprised. This is contributed by means of a reducing agent containing a thiol moiety. Thioglycolic Acid (TGA) @ 5% concentration is the preferred embodiment. Other agents, such as Dithiothretol (DTT), β-Mercaptoethanol (β-ME) and Urea Hydrogen Peroxide @ 17.5% concentration might be similarly employed to act upon the hydrogen bonds, as well as the disulfide bonds.

An additional keratolytic agent or enzyme, such as Proteinase K might be employed to degrade the keratin substrate @ about 10 mg/mL. The optimal pH of keratolytic activity is around pH 8, while activity is detected in a broad range of pH values between 6 to 11 for serine proteases. Chemical hydrolysis will further compromise the barrier property contributed by the corneocytes but the process is irreversible and concentration-dependent.

Suitable proteases for use in embodiments of the invention, including as targets for inhibition, are described in U.S. Pat. No. 8,211,428 by Madison, E. L. entitled “Protease screening methods and protease identified thereby’, U.S. Pat. No. 9,458,374 by Sorrells, D. D. entitled ‘Cysteine proteases for bacterial control’, Powers, J. C., et al., Irreversible inhibitors of serine, cysteine, and threonine proteases, Chem. Rev., 2002, 102 (12), pp 4639-4750; Turk B. et al., ‘Regulating Cysteine Protease Activity: Essential Role of Protease Inhibitors As Guardians and Regulators’, Curr. Pharma Des., V8, 18, 2002, DOI:10.2174/1381612023394124; all incorporated by reference herein.

The simultaneous application of the reducing agent has been demonstrated to have no adverse effect on the keratolytic enzymes and, in fact, allows the preferential access of the enzymes to the substrate for enhanced proteolytic attack.

Sigma-Aldrich offers an appropriate keratinolytic product (K4519-500UN), which is a non-specific serine protease with the capability of degrading insoluble keratin substrates by cleaving non-terminal peptide bonds.

This patent further embodies an alternative to the reducing agent/keratolytic enzyme combination by means of two cooperating enzymes isolated from a keratin-degrading bacterium, Stenotrophomonas sp. strain D-1. These synergistic enzymes disrupt the disulfide bonds while simultaneously degrading the keratin substrate.

### Formulations

Enhancement of transdermal drug delivery directed to the cellular component of the SC barrier is a complex process and, therefore might employ individual CPEs or mixtures of chemicals.

The formulations comprise mixtures wherein the CPEs interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers. (As used herein “detergent” and “surfactant” are synonymous).

The preferred biochemical process, which is directed to the extra-cellular lipid matrix of the SC permeability barrier and is facilitated by the carrier, which preferentially employs additional penetrants described in the cited US2009/0053290 ('290) and WO2014/209910 ('910)—i.e., benzyl alcohol and a lecithin organogel, but at much higher ratios of lecithin organogel to benzyl alcohol than in the prior art compositions. The present carriers also may include a nonionic surfactant which is disclosed to be undesirable in the '910 publication and is described in the '290 publication as present only in very low amounts. The applicant has found that by employing very high amounts of the lecithin organogel relative to benzyl alcohol and relative to the weight of the formulation, as well as in some embodiments providing a combination of a nonionic surfactant and molar excess of a polar gelling agent, the penetration capabilities of the resulting formulation and the effective level of delivery of the active agent can be greatly enhanced. Such a result was completely unpredictable as it was believed that relatively equal amounts of the benzyl alcohol and lecithin organogel especially a somewhat higher concentration of benzyl alcohol than lecithin organogel were responsible for the level of penetration achieved by prior art formulations.

Water-in-oil microemulsions have a generic role in the delivery of a wide range of water-soluble molecules from 100 to 150 kDa. The bio-activity is maintained during formulation with microemulsions and during transit through the skin.

Soy lecithin phosphotidylcholine has been revealed to form a noncovalent complex with TD-1, which implies an interaction between TD-1 and the negatively charged cell lipids. Microemulsions consisting of bile salts, lecithin organogel and electrolytes have been used to form supramolecular structure that can increase not only skin permeability but also drug solubility in formulation and drug partitioning into the skin.

Lecithin is a biosurfactant and a zwitterionic phospholipid molecule with a head group having a positively charged choline and a negatively charged phosphate. When a small quantity of water is added to these fluids, the lecithin tends to self-organize into bi-layer membranes and in turn into vesicles or spherical micelles. Water is the most commonly employed polar agent although some other polar agents such as glycerol, ethylene glycol and formamide have been found to possess the capability of transferring an initial non-viscous lecithin solution into a jelly-like state.

The first examples of such micelles were tertiary mixtures of lecithin-water-oil (organic solvents). While lecithin alone forms vesicles or micelles, these micelles are inherently unstable because the bulky hydrophobic tails of the lipid (lecithin) inhibit its solubility in water.

The lecithin organogel included in the composition is a combination of lecithin with an organic solvent, which is typically amphiphilic. Suitable organic solvents include, in addition to isopropyl palmitate, ethyl laurate, ethyl myristate and isopropyl myristate. Certain hydrocarbons, such as cyclopentane, cyclooctane, trans-decalin, trans-pinane, n-pentane, n-hexane, n-hexadecane may also be used. The ratio of lecithin to isopropyl palmitate may be 50:50. For examples, a formulation containing soy lecithin in combination with isopropyl palmitate is employed, however, other lecithins could also be used such as egg lecithin or synthetic lecithins. Soy lecithin comprised of 96% pure phosphatidylcholine is preferred.

Various esters of long chain fatty acids may also be included. Methods for making such lecithin organogels are well known in the art. In most embodiments, the lecithin organogel is present in the final formulation in the range of 25-70% w/w and at intermediate percentages such as 30% w/w, 40% w/w, 50% w/w, 60% w/w, etc.

Lecithin organogels may be in the form of vesicles, microemulsions and micellar systems. In the form of self-assembled structures, such as vesicles or micelles, they can fuse with the lipid bilayers of the stratum corneum, thereby enhancing partitioning of encapsulated drug, as well as a disruption of the ordered bilayers structure. An example of a phospholipid-based permeation enhancement agent comprises a micro-emulsion-based organic gel defined as a semisolid formation having an external solvent phase immobilized within the spaces available of a three-dimensional networked structure. This micro-emulsion-based organic gel in liquid phase is characterized by 1,2-diacyl-sn-glycero-3-phosphatidyl choline, and an organic solvent, which is at least one of: ethyl laureate, ethyl myristate, isopropyl myristate, isopropyl palmitate; cyclopentane, cyclooctane, trans-decalin, trans-pinane, n-pentane, n-hexane, n-hexadecane, and tripropylamine.

Lecithin microemulsion-based organogels are thermodynamically stable, clear, visco elastic, biocompatible and isotropic phospholipid structured systems. The naturally occurring surfactant, lecithin, can form reverse micelle-based microemulsions in non-polar environment because of its geometric discipline. These small reverse micelles upon addition of a specific amount of water, likely grow mono-dimensionally into long flexible and cylindrical giant micelles, above a critical concentration of lecithin. These giant micelles form a continuous network that immobilizes the external organic phase forming a gel or jelly-like state.

The applicant has further discovered that, while lecithin alone forms vesicles or micelles, these micelles are inherently unstable. The addition of second class of biosurfactants, bile salts, in small amounts will intercalate into lecithin vesicles and stabilize these structures. Accordingly, lecithin-bile salt vesicles have been examined in the context of lipid-protein interactions.

Alternatively, an anhydrous composition may be obtained by using, instead of a polar component, a material such as a bile salt. When formulated with bile salts, the micellular rheologic nature of the composition is altered so that rather than a more or less spherical vesicular form, the vesicles become wormlike and are able to accommodate larger guest molecules, as well as penetrate the epidermis more effectively.

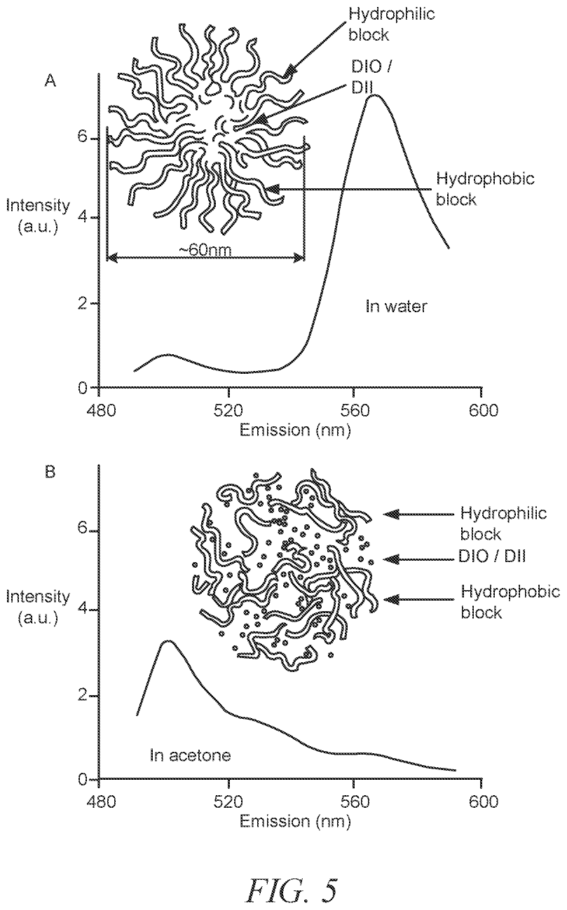

The effective transdermal delivery of drugs is dependent upon three critical factors involved in the self-assembly of micelles; thermodynamic and kinetic stability viscosity and viscoelasticity. Lecithin organogel micelles are inherently unstable, thereby releasing their cargo prematurely before reaching the target site. The introduction of bile salts result in enhanced micellar stability (FIG. 5), viscosity and visco-elasticity. Suitable bile salts include salts of deoxycholic acid, taurocholic acid, glycocholic acid, taurochenodeoxycholic acid, glycochenodeoxycholic acid, cholic acid and the like. Certain detergents, such as Tween® 80 or Span® 80 may be used as alternatives.

The applicant has additionally discovered that the formation of worms also requires a background electrolyte at sufficient levels. These electrolytes, such as sodium citrate, are required to more effectively increase viscosity and visco-elasticity of micelles and screen the repulsion between bile salt anions at a minimal concentration. Another effect of sodium citrate is its ability to “salt out” solutes from water as the Hofmeister effect. In other words, a specific molar ratio and a sufficient electrolyte concentration are required for the formation of stable, long flexible cylindrical micelles.

The percentage of these components in the anhydrous forms of the composition is in the range of 1% w/w-15% w/w. In some embodiments, the range of bile salt content is 2%-6% w/w or 1%-3.5% w/w. In these essentially anhydrous forms, powdered or micronized nonionic detergent is used to top off, typically in amounts of 20%-60% w/w. In one approach to determine the amount of bile salt, the % is calculated by dividing the % w/w of lecithin by 10.

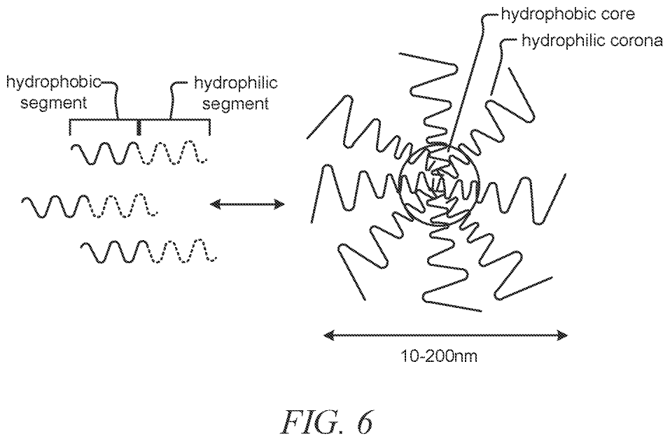

It is now widely recognized that these bile salt-stabilizing vesicles are very similar to polymeric chains with the important exception that these vesicles are in thermal equilibrium with their monomers and break and recombine at a rapid rate. A competition between vesicular breaking and chain reputation dictates the rheology of the fluid. Recent studies have focused on the role played by the water in reverse micellar growth (water can be substituted with other polar solvents, such as glycerol). These studies have yielded disparate and sometimes diverging conclusions; some have speculated that water is the necessary glue that holds these reverse micelles together, but this has been refuted by others. These spherical vesicles grow axially into flexible cylinders, thus, the crucial component is water and the molar ratio of water to lecithin (denoted by w₀) is the key parameter in dictating micellar growth (FIG. 6).

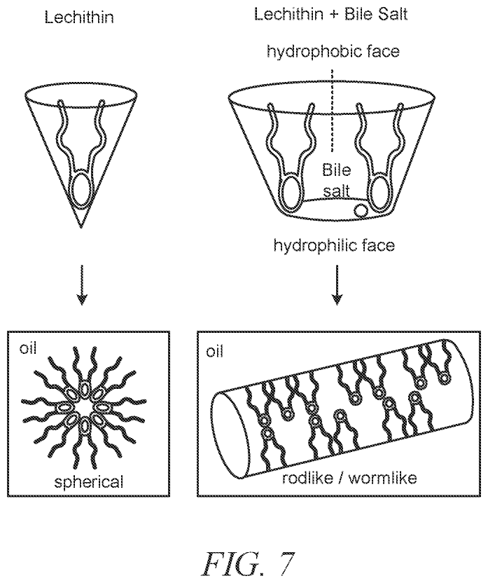

In embodiments where a bile salt is added to the combination of benzyl alcohol and lecithin organogel in lieu of topping off with an aqueous medium, micelles that would have been relatively spherical may become elongated and worm-like thus permitting superior penetration of the stratum corneum of the epidermis (FIG. 7). The worm like formation of the micelles is particularly helpful in accommodating higher molecular weight therapeutic agents. Bile salts are facial amphiphiles and include salts of taurocholic acid, glycocholic acid, taurochenodeoxycholic acid, glycochenodeoxycholic acid, cholic acid, deoxycholic acid. Other detergents are also useful in lieu of bile salts, and include Tween® 80 and Span® 80.

The inclusion of these bile salts facilitates the ultradeformability of micelles which, in turn, facilitate passage of low and high molecular weight drugs and other active agents, such as nucleic acids and proteins. These compositions overcome the skin penetration barrier by squeezing themselves along the intercellular sealing lipid thereby following the natural gradient across the stratum corneum. This facilitates a change in membrane composition locally and reversibly when pressed against or attracted to a narrow pore.

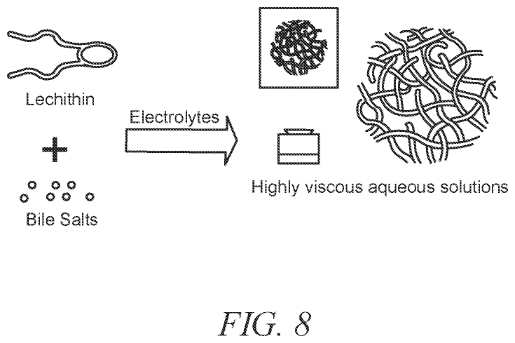

Bile salts in combination with lecithin organogel facilitate the factors of micellar stability, enhanced viscosity and visco-elasticity so critical in transdermal drug delivery. Both thermodynamic and kinetic stability is enhanced by the addition of background electrolytes, such as sodium chloride and sodium citrate (FIG. 8). Sodium citrate is the more effective electrolyte because it is strongly ionic, thereby reinforcing the interactions between water molecules and various solutes. These electrolytes can more effectively increase viscosity and visco-elasticity of micelles and screen the repulsion between bile salt anions at a minimal concentration.

The molar ratio of bile salt to lecithin is 1:1, but the concentration of electrolyte is determined by titration of the solution to transparency of the solution and enhanced viscosity as determined when the solution container is inverted.

In some formulations of the invention, in addition to the above amounts of bile salts, benzyl alcohol, lecithin organogel and active ingredient, the formulations are “topped off” with a powdered nonionic detergent. The pH of such compositions can be determined by taking a small sample and dissolving it in water to test the appropriate pH. In many embodiments, the pH is in the range of 8.5-11 or 9-11 or 10-11.

An additional required component in the formulations of the invention is an alcohol. Benzyl alcohol in some formulations but other alcohols could be included, in particular derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 5% w/w, 7% w/w, 10% w/w, and other intermediate weight percentages are included.

Due to the aromatic group present in a permeation enhancement formulation such as benzyl alcohol, the molecule has a polar end (the alcohol end) and a non-polar end (the benzene end). This enables the agent to dissolve a wider variety of drugs and agents. The alcohol concentration is substantially lower than the concentration of the lecithin organogel in the composition.

By formulating active ingredients in the presence of at least a combination of a lecithin organogel and a suitable alcohol, especially benzyl alcohol where the lecithin organogel is in a ratio of concentration at least 10-fold that of the alcohol on a weight basis, superior results are achieved as illustrated in the examples below.

In some embodiments, as noted above, the performance of the formulations is further improved by including a nonionic detergent and polar gelling agent or including bile salts and a powdered surfactant. In both aqueous and anhydrous forms of the composition, detergents, typically nonionic detergents are added. In general, the nonionic detergent should be present in an amount of at least 2% w/w to 60% w/w. Typically, in the compositions wherein the formulation is topped off with a polar or aqueous solution containing detergent, the amount of detergent is relatively low—e.g., 2%-25% w/w, or 5-15% w/w or 7-12% w/w.

However, in compositions comprising bile salts that are essentially anhydrous and are topped-off by powdered detergent, relatively higher percentages are usually used—e.g., 20%-60% w/w. The boundaries are not rigid but the above description indicates the general range.

In some embodiments, the nonionic detergent provides suitable handling properties whereby the formulations are gel-like or creams at room temperature. To exert this effect, the detergent, typically a poloxamer, is present at a level of at least 9% w/w, preferably at least 12% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohol and copolymers of hydrophilic and hydrophobic monomers where blocks of hydrophilic and hydrophobic portions are used.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by SigmaAldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like.

In the presence of a polar gelling agent, such as water, glycerol, ethylene glycol or formamide, a micellar structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition. As noted above, it may be rather than a polar gelling agent, a bile salt can be used. In this case, the detergent is added in solid, powdered form.

The percentage of active agent in the formulation will depend upon the concentration required to be delivered in order to have a useful effect on treating the disorder. In general, the active ingredient may be present in the formulation in an amount as low as 0.01% w/w up to about 50% w/w. Typical concentrations include 0.25% w/w, 1% w/w, 5% w/w, 10% w/w, 20% w/w and 30% w/w. Since the required percentage of active ingredient is highly variable depending on the active agent and depending on the frequency of administration, as well as the time allotted for administration for each application, the level of active ingredient may be varied over a wide range, and is limited only by the necessity for including in the formulation aids in penetration of the skin by the active ingredient.

The formulations of the invention may include only one active agent or a combination of active agents. In the present application, “active agent” or “active ingredient” refers to a compound or drug that is active against the factors or agents that result in the desired therapeutic or other localized systemic effect.

In general, in the present application, “a,” “an,” “one,” and the like should be interpreted to mean one or more than one unless it is clear from the context that only a single referent is intended. Thus, “an active ingredient” may refer to one or more such active ingredients.

The formulations of the invention may be prepared in a number of ways. Typically, the components of the formulation are simply mixed together in the required amounts. However, it is also desirable in some instances to, for example, carry out dissolution of an active ingredient and then add a separate preparation containing the components aiding the delivery of the active ingredients in the form of a carrier. The concentrations of these components in the carrier, then, will be somewhat higher than the concentrations required in the final formulation

Alternatively some subset of these components can first be mixed and then “topped off” with the remaining components either simultaneously or sequentially. The precise manner of preparing the formulation will depend on the choice of active ingredients and the percentages of the remaining components that are desirable with respect to that active ingredient.

As noted above, the essential components of the formulations for most applications are 25%-70% w/w lecithin organogel and 0.5-20% w/w benzyl alcohol or closely related alcohol as well as supplementary components such as detergents, typically nonionic detergents, bile salts, polar solvents and the like.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature otherwise stated. The concentration of hydroxypropylcellulose may range from about 1% w/w to about 2% w/w of the composition. Other gelling agents are known in the art and can be used in place of, or in addition to, hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Typical components that may also be included in the formulations are fatty acids, terpenes, lipids, and cationic and anionic detergents.

Other solvents and related compounds that may be used in some embodiments include acetamide and derivatives, acetone, n-alkanes (chain length between 7 and 16), alkanols, diols, short-chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefossé, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C3-C6 triols or their mixtures and a polar lipid compound selected from C16 or C18 monounsaturated alcohol, C16 or C18 branched saturated alcohol and their mixtures, propylene glycol, sorbitan monolaurate sold as Span® 20 sold by Sigma-Aldrich, squalene, triacetin, trichloroethanol, trifluoroethanol, trimethylene glycol and xylene.

Fatty alcohols, fatty acids, fatty esters, are bilayer fluidizers that may be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol.

Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monolaurate, sorbitan trilaurate, sorbitan trioleate, sucrose coconut fatty ester mixtures, sucrose monolaurate, sucrose monooleate, tetradecyl N,N-dimethylamino acetate.

Examples of suitable fatty acid include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid.

Examples of suitable fatty alcohol ethers include α-monoglyceryl ether, E0-2-oleyl ether, E0-5-oleyl ether, E0-10-oleyl ether, ether derivatives of polyglycerols and alcohols, and (1-O-dodecyl-3-O-methyl-2-0-(2′,3′dihydroxypropyl)glycerol).

Examples of completing agents that may be used in some embodiments include β- and γ-cyclodextrin complexes, hydroxypropyl methylcellulose (such as Carbopol® 934), liposomes, naphthalene diamide diimide, and naphthalene diester diimide.

One or more anti-oxidants may be included, such as vitamin C, vitamin E, proanthocyanidin and α-lipoic acid typically in concentrations of 0.1%-2.5% w/w.

In some applications, it is desirable to adjust the pH of the formulation to assist in permeation or to adjust the nature of the active agent and/or of the target compounds in the subject. In some instances, the pH is adjusted to a level of pH 9-11 or 10-11 which can be done by providing appropriate buffers or simply adjusting the pH with base.

Skin's electrical resistance or impedance is generally considered a marker of skin permeability and changes in skin resistance due to exposure to different CPEs has been shown to correlate with increased skin permeability to model drug compounds. From a mechanistic viewpoint, skin's electrical resistance is known to be governed primarily to the highest ordered, lipophilic barrier of the SC lipid bilayers. Therefore, changes in skin's resistance are a sensitive measure of changes in the SC lipid bilayer integrity. Changes in skin's resistance are seen to occur with a lag time of one or more hours, which suggests a kinetic barrier that may be a diffusive transport limitation.

Measurement of skin's resistance or impedance can be used to as a ‘generic’ measurement of skin permeability that does not depend on the specific characteristics of target molecules, such as hydrophobicity and charge.

The electrical resistance or impedance across the epidermis was measured as the CPE was applied to the skin, which was maintained at 32° C. Electrical conductivity was calculated from electrical resistance measurements.

In some formulations, formation of micelles is enhanced by milling. The level of enhancement is determined by the pressure and speed at which milling occurs as well as the number of passes through the milling machine. As the number of passes and the pressure is increased, the level of micelle formulation is enhanced as well. In general, increasing the pressure and increasing the speed of milling enhances the level of micelle density.

When the ointment milling machine is a Dermamill 100 (Blaubrite) marketed by Medisca®, typical speeds include any variation between 1 to 100, where 1 is the slowest speed and 100 is the fastest speed, such as speeds of 1, 5, 10, 15, 20, 25, 30, 35, 40, 45, 50, 55, 60, 65, 70, 75, 80, 85, 90 or 100, or any speed in between. The pressure is selected from 1 to 5, where 1 is the highest pressure and 5 is the lowest pressure. The pressure used can be selected from 1, 2, 3, 4, or 5. The number of passes can also be varied, where a pass is complete when all of the product has passed through the rollers of the machine. Multiple passes, such as 2, 3, 4, 5, 6, 7, 8, 9, 10 or more passes, are contemplated in some embodiments. The speed and pressure can be varied for each pass. For example, a first pass may have a first pressure and first speed, while a second (or subsequent) pass may have a second pressure and second speed, where the second pressure is the same or different from the first pressure and the second speed is the same or different from the first speed. The desired micelle density for particular formulations can be determined empirically by varying the speed, pressure and number of passes.

Of course, alternative ointment milling machines could also be used, and comparable speeds, pressures and numbers of passes are replicated by comparison to the equivalents on the Dermamill 100. Alternatively, micelle densities can be compared microscopically to assure equivalent results to those set forth herein. In some embodiments, the micelle density is at least 20% and in many cases at least 30%, 50%, 70%, 80% or 90% and all levels within this range.

These studies have yielded specific and rare binary mixtures of CPEs in synergistic combination that enhance skin permeability to hydrophilic macromolecules by more than 50-fold without inducing skin irritation.

The preferred embodiment of this patent is an integrative cooperative formulation combining: (1) binary mixtures of CPEs selected from electrometric screening, (2) dual biosurfactant-based reverse wormlike-micellar systems, (3) bipolar aliphatic alcoholic solvents, (4) keratinolytic agents, (5) thiol-moiety reducing agents, and (6) skin penetrating peptides (SPPs) in a higher ordered topical transdermal drug delivery composition, which effectively hosts various guest drug molecules, thereby, breaching the SC permeation barrier and rendering them bioavailable to their target site.

This is an example of an integrative cooperative CPE formulation directed to the extra-cellular matrix to which might be added selected cysteine cathepsin protease-inhibitors, with or without a suitable buffering agent.

1. Cetyltrimethyl ammonium bromide (from about 2.0% to about 10.0%)

2. Sodium cholate: Lecithin (96% pure): Isopropyl myristate (equi-molar 1:1:1 (from about 10% to about 40.0%)

3. Sodium citrate (titrate to transparency/incr. viscosity of #2.)

4. Benzyl alcohol (from about 2.0% to about 30.0%)

5. Cis-Palmitoleic acid (from about 20.0% to about 30% of BA)

6. Methyl pyrrolidone (0.4%)/Dodecyl pyridinium (1.1%) (from about 0.5% to about 5.0%)

7. Pluronic 127 (qs to 100%)

This is an example of the formulation, which is directed to the cellular component of the SC permeability barrier to which might be added selected cysteine cathepsin protease-inhibitors, with or without a suitable buffering agent.

1. ACSSSPSKHCG, [alanine-cysteine-serine-serine-serine-proline-serine-lysine-hisitidine-cysteine-glycine] identified as TD-1

2. Thioglycolic Acid (TGA) (from about 2.0% to about 7.0% concentration) [may be substituted by other reducing agents]

3. Proteinase K (from about 5 mg/mL to about 15 mg/mL)

A formulation for transdermal delivery may, for example, comprise two components or it may comprise one or more buffering agent and a penetrant. Typically, however, a penetrant is less than 85% w/w. The formulation may have a detergent of at least 1% w/w. For example, a suitable formulation may comprise about 10-56% w/w buffering agent and a penetrant. In one aspect, disclosed herein is a formulation for transdermal delivery of one or more buffering agent through the skin of a subject, comprising: a buffering agent comprising a carbonate salt in an amount between about 10-56% w/w; a penetrant portion in an amount between about 5 to 55% w/w; a detergent portion in an amount of at least 1% w/w; and wherein the formulation comprises water in an amount from none up to about 77% w/w.

In another aspect, disclosed herein is a method for transdermal delivery of a carbonate salt of the formulation comprising: a buffering agent comprising a carbonate salt in an amount between about 10-45% w/w; a penetrant portion in an amount between about 5 to 55% w/w; a detergent portion in an amount between about 1 to 15% w/w; and wherein the formulation comprises water in an amount between about 15 to 65% w/w, through the skin of a subject, wherein the carbonate salt of the formulation is in an amount between about 15-32% w/w of the formulation.

In yet another aspect, disclosed herein is a formulation for transdermal delivery of a therapeutic agent through the skin of a subject, wherein the formulation comprises at least one active agent in an amount effective for treatment of a condition in the subject and the formulation comprising: a buffering agent comprising a carbonate salt in an amount between about 10-45% w/w; a penetrant portion in an amount between about 5 to 55% w/w; a detergent portion in an amount between about 1 to 15% w/w; wherein the formulation comprises water in an amount between about 15 to 65% w/w, through the skin of a subject, wherein the carbonate salt of the formulation is in an amount between about 15-32% w/w of the formulation, therapeutic, and wherein the alkalinity of the formulation enhances penetration of the therapeutic agent.

In one aspect, disclosed herein is a formulation for transdermal delivery of one or more buffering agent through the skin of a subject, comprising: a buffering agent comprising a carbonate salt in an amount between about 10-45% w/w; a penetrant portion in an amount between about 5 to 55% w/w; a detergent portion in an amount between about 1 to 15% w/w; and wherein the formulation comprises water in an amount between about 15 to 65% w/w, and wherein the formulation comprises less than about 12% w/w lecithin.

In another aspect, disclosed herein is a method for transdermal delivery of a carbonate salt of the formulation comprising: a buffering agent comprising a carbonate salt in an amount between about 10-45% w/w; a penetrant portion in an amount between about 5 to 55% w/w; a detergent portion in an amount between about 1 to 15% w/w; and wherein the formulation comprises water in an amount between about 15 to 65% w/w, and wherein the formulation comprises less than about 12% w/w lecithin, through the skin of a subject, wherein the carbonate salt of the formulation is in an amount between about 15-32% w/w of the formulation, wherein the formulation comprises less than about 12% w/w lecithin, and wherein the alkalinity of the formulation enhances penetration of the therapeutic agent.

In yet another aspect, disclosed herein is a formulation for transdermal delivery of a therapeutic agent through the skin of a subject, wherein the formulation comprises at least one active agent in an amount effective for treatment of a condition in the subject and the formulation comprising: a buffering agent comprising a carbonate salt in an amount between about 10-45% w/w; a penetrant portion in an amount between about 5 to 55% w/w; a detergent portion in an amount between about 1 to 15% w/w; wherein the formulation comprises water in an amount between about 15 to 65% w/w, through the skin of a subject, wherein the carbonate salt of the formulation is in an amount between about 15-32% w/w of the formulation, and wherein the formulation comprises less than about 12% w/w lecithin.

In some embodiments, a suitable formulation comprises: Lipmax™ in an amount between about 1-20% w/w; benzyl alcohol in an amount between about 0.25 to 5% w/w; menthol in an amount between about 0.1-5% w/w; Pluronic® in an amount between about 0.1-5% w/w; water in an amount between about 10-80% w/w; sodium carbonate in an amount between about 1-32% w/w; sodium bicarbonate in an amount between about 1-32% w/w; ethylene glycol tetraacetic acid in an amount less than about 5% w/w; propylene glycol in an amount between about 0.5-10% w/w; almond oil in an amount between about 0.5-10% w/w; cetyl alcohol in an amount between about 0.5-10% w/w; lecithin in an amount less than about 12% w/w; Cetiol Ultimate® in an amount less than about 10% w/w; and ethanol in an amount between about 0.5-10% w/w.

In some embodiments, a suitable formulation comprises: Lipmax™ in an amount between about 1-20% w/w; benzyl alcohol in an amount between about 0.25 to 5% w/w; menthol in an amount between about 0.1-5% w/w; Durasoft® in an amount between about 0.1-5% w/w; Pluronic® in an amount between about 0.1-5% w/w; water in an amount between about 10-80% w/w; sodium carbonate in an amount less than about 32% w/w; sodium bicarbonate in an amount between about 1-32% w/w; ethylene glycol tetraacetic acid in an amount less than about 5% w/w; sodium decanoate in an amount less than about 5% w/w; propylene glycol in an amount between about 0.5-10% w/w; almond oil in an amount between about 0.5-10% w/w; zinc oxide in an amount less than about 2% w/w; cetyl alcohol in an amount between about 0.5-10% w/w; and ethanol in an amount between about 0.5-10% w/w.

In some embodiments, a suitable formulation comprises: Water in an amount between about 10-80% w/w; Phospholipon® 90G in an amount between about 0.5-16% w/w; Myritol® 312 in an amount between about 0.5-10% w/w; isopropyl palmitate in an amount between about 1-10% w/w; Cetiol® Ultimate in an amount between about 0.25-5% w/w; stearic acid in an amount between about 0.25-5% w/w; cetyl alcohol in an amount between about 0.25-5% w/w; benzyl alcohol in an amount between about 0.25-5% w/w; propylene glycol in an amount between about 0.25-5% w/w; glycerin in an amount between about 0.25-5% w/w; ethanol in an amount between about 0.25-5% w/w; Pluronic® in an amount between about 0.1-5% w/w; Lipmax™ in an amount between about 1-20% w/w; and sodium bicarbonate in an amount between about 1-32% w/w.

In some embodiments, a suitable formulation comprises: Siligel™ in an amount between about 1-5% w/w; water in an amount between about 10-80% w/w; Phospholipon® 90G in an amount between about 0.5-16% w/w; Myritol® 312 in an amount between about 0.5-10% w/w; isopropyl palmitate in an amount between about 1-10% w/w; Cetiol® Ultimate in an amount between about 0.25-5% w/w; stearic acid in an amount between about 0.25-5% w/w; cetyl alcohol in an amount between about 0.25-5% w/w; benzyl alcohol in an amount between about 0.25-5% w/w; propylene glycol in an amount between about 0.25-5% w/w; glycerin in an amount between about 0.25-5% w/w; ethanol in an amount between about 0.25-5% w/w; sodium hydroxide 50% w/v in an amount between about 0.1-5% w/w; Lipmax™ in an amount less than about 20% w/w; and sodium bicarbonate in an amount between about 1-32% w/w.

In some embodiments, a suitable formulation comprises: water in an amount between about 10-80% w/w; Phospholipon® 90G in an amount between about 0.5-10% w/w; Myritol® 312 in an amount between about 0.5-10% w/w; isopropyl palmitate in an amount between about 0.5-10% w/w; Cetiol® Ultimate in an amount less than about 10% w/w; stearic Acid in an amount between about 0.25-5% w/w; cetyl alcohol in an amount between about 0.25-5% w/w; benzyl alcohol in an amount between about 0.25-5% w/w; propylene glycol in an amount between about 0.25-5% w/w; glycerin in an amount between about 0.25-5% w/w; ethanol in an amount between about 0.25-5% w/w; sodium hydroxide 50% w/v in an amount between about 0.1-5% w/w; and sodium bicarbonate in an amount between about 1-35% w/w.

In some embodiments, a suitable formulation comprises: water in an amount between about 10-40% w/w; Phospholipon® 90H in an amount between about 0.5-20% w/w; Myritol® 312 in an amount between about 0.5-10% w/w; isopropyl palmitate in an amount between about 0.5-20% w/w; Cetiol® Ultimate in an amount less than about 10% w/w; stearic acid in an amount between about 0.25-5% w/w; cetyl alcohol in an amount between about 0.25-5% w/w; benzyl alcohol in an amount between about 0.25-5% w/w; propylene glycol in an amount between about 0.25-5% w/w; glycerin in an amount between about 0.25-5% w/w; ethanol in an amount between about 0.25-5% w/w; sodium hydroxide 50% w/v in an amount between about 0.1-5% w/w; and sodium bicarbonate in an amount between about 1-35% w/w.

In some embodiments, a suitable formulation comprises: water in an amount between about 10-40% w/w; Phospholipon® 90H in an amount between about 0.5-20% w/w; Phospholipon® 90G in an amount between about 0.5-20% w/w; Myritol® 312 in an amount between about 0.5-10% w/w; isopropyl palmitate in an amount between about 0.5-20% w/w; Cetiol® Ultimate in an amount less than about 10% w/w; stearic acid in an amount between about 0.25-5% w/w; cetyl alcohol in an amount between about 0.25-5% w/w; benzyl alcohol in an amount between about 0.25-5% w/w; propylene glycol in an amount between about 0.25-5% w/w; glycerin in an amount between about 0.25-5% w/w; ethanol in an amount between about 0.25-5% w/w; sodium hydroxide 50% w/v in an amount between about 0.1-5% w/w; and sodium bicarbonate in an amount between about 1-35% w/w.

In some embodiments, a suitable formulation comprises: water in an amount between about 10-50% w/w; Pluronic® gel 30% in an amount between about 5-30% w/w; isopropyl palmitate in an amount between about 0.5-20% w/w; stearic Acid in an amount between about 0.25-10% w/w; cetyl alcohol in an amount between about 0.25-10% w/w; benzyl alcohol in an amount between about 0.25-5% w/w; almond oil in an amount between about 0.5-10% w/w; propylene glycol in an amount between about 0.25-10% w/w; ethanol in an amount between about 0.25-5% w/w; sodium hydroxide 50% w/v in an amount between about 0.1-5% w/w; and sodium bicarbonate in an amount between about 1-32% w/w.

In some embodiments, a suitable formulation comprises: Siligel™ in an amount less than about 5% w/w; water in an amount between about 10-65% w/w; isopropyl palmitate in an amount between about 0.5-10% w/w; stearic Acid in an amount between about 0.25-10% w/w; cetyl alcohol in an amount between about 0.25-10% w/w; glycerin in an amount between about 0.25-5% w/w; Lipmax™ in an amount between about 0.25-10% w/w; ethanol in an amount less than about 5% w/w; benzyl alcohol in an amount less than about 5% w/w; sodium hydroxide 50% w/v in an amount between about 0.1-5% w/w; and sodium bicarbonate in an amount between about 1-32% w/w.

In some embodiments, a suitable formulation comprises: Aveeno® in an amount between about 20-85% w/w; and sodium bicarbonate (3DF) in an amount between about 15-45% w/w.

In some embodiments, a suitable formulation comprises: Aveeno® in an amount between about 20-85% w/w; and sodium bicarbonate (Milled #7) in an amount between about 15-45% w/w.

In some embodiments, a suitable formulation comprises: Siligel™ in an amount less than about 5% w/w; water in an amount between about 10-55% w/w; isopropyl palmitate in an amount between about 0.5-10% w/w; stearic Acid in an amount between about 0.25-5% w/w; Cetyl alcohol in an amount between about 0.25-10% w/w; almond oil in an amount between about 0.5-10% w/w; propylene glycol in an amount between about 0.25-10% w/w; ethanol in an amount less than about 5% w/w; benzyl alcohol in an amount less than about 5% w/w; sodium hydroxide 50% w/v in an amount between about 0.1-5% w/w; and sodium bicarbonate in an amount between about 1-32% w/w.

The surprising effects achieved by the formulations and methods of the present invention are in part attributable to an improved formulation that enhances delivery of a carbonate salt through the skin. In some embodiments, the formulation employs penetrants described US2009/0053290 (′290), WO2014/209910 (′910), and WO2017/127834. The present formulations may include a nonionic surfactant.

Applicant has found that by employing carbonate salts with particle sizes as disclosed herein, delivered with the penetrants as disclosed herein, and in some embodiments providing a combination of a nonionic surfactant and a polar gelling agent, the penetration capabilities of the carbonate salts of the resulting formulation and the effective level of delivery of the carbonate salts has been enhanced. This enhanced level of penetration was also achieved using significantly less lecithin than anticipated or none at all. This result was completely unexpected as it was believed that relatively equal amounts of the benzyl alcohol and lecithin organogel especially a somewhat higher concentration of benzyl alcohol than lecithin organogel were responsible for the level of penetration achieved by prior art formulations.

Briefly, the penetrants described in the above-referenced US and PCT applications are based on combinations of synergistically acting components. Many such penetrants are based on combinations of an alcohol, such as benzyl alcohol to provide a concentration of 0.5-20% w/w of the final formulation with lecithin organogel present in the penetrant to provide 25-70% w/w of the formulation. These penetrants are also useful when the agent is a buffer, such as sodium bicarbonate, but less lecithin organogel may be required—e.g. less than 12% w/w when the sodium bicarbonate is present at high concentration as disclosed herein.

In some embodiments, the buffering component is any mildly basic compound or combination that will result in a pH of 7-8 in the microenvironment of the tumor cells. In some embodiments, the formulation has a pH of 7-10. Such buffers, in addition to carbonate and/or bicarbonate salts, include lysine buffers, chloroacetate buffers, tris buffers (i.e., buffers employing tris (hydroxymethyl) aminoethane), phosphate buffers and buffers employing non-natural amino acids with similar pKa values to lysine. In some embodiments, the carbonate and/or bicarbonate salt is in an amount between about 7-32% w/w of the formulation. For example, the enantiomers of native forms of such amino acids or analogs of lysine with longer or shorter carbon chains or branched forms thereof. Histidine buffers may also be used. Typically, the concentration of buffer in the compositions is in the range of 10-50% w/w. More typical ranges for sodium bicarbonate or sodium carbonate or both are 10-35% by weight. In some embodiments, the carbonate salt is in an amount between about 15-32% w/w of the formulation.

Alternatively, the penetrant component comprises a completion component as well as one or more electrolytes sufficient to impart viscosity and viscoelasticity, one or more surfactants and an alcohol. The completion component can be a polar liquid, a non-polar liquid or an amphiphilic substance.

The percentage of carbonate salt in the formulation will depend upon the amount required to be delivered in order to have a useful effect on treating the disorder. In general, the carbonate salt may be present in the formulation in an amount as low as 1% w/w up to about 50% w/w. Typical concentrations may include 15-32% w/w. Since the required percentage of carbonate salt depends on the frequency of administration, as well as the time allotted for administration for each application, the level of carbonate salt may be varied over a wide range. In some embodiments, the carbonate salt is sodium carbonate and/or sodium bicarbonate milled to a particle size is less than 200 μm. In some embodiments, the carbonate salt is sodium carbonate and/or sodium bicarbonate milled to a particle size is less than 70 μm. In some embodiments, the carbonate salt is sodium carbonate and/or sodium bicarbonate milled to a particle size is less than 70 μm, wherein the sodium bicarbonate is solubilized in the formulation in an amount less than 20% w/w of the formulation. In some embodiments, the carbonate salt is sodium carbonate and/or sodium bicarbonate milled to a particle size is less than 70 μm, wherein particle sizes less than about 10 μm have an enhanced penetration thru the skin of a subject. In some embodiments, the sodium carbonate and/or sodium bicarbonate are jet milled to a particle size less than about 70 μm. In some embodiments, the sodium bicarbonate is Sodium Bicarbonate USP Grade 3DF that has a particle size distribution less than 70 μm.

The formulations of the disclosure may be prepared in a number of ways. Typically, the components of the formulation are simply mixed together in the required amounts. However, it is also desirable in some instances to, for example, carry out dissolution of a carbonate salt and then add a separate preparation containing the components aiding the delivery of the carbonate salts in the form of a carrier. The concentrations of these components in the carrier, then, will be somewhat higher than the concentrations required in the final formulation. Thus, sodium bicarbonate may first be dissolved in water and then added to a carrier comprising an alcohol, lecithin and optionally a combination of a nonionic surfactant and polar gelling agent, or of ionic detergent. Alternatively, some subset of these components can first be mixed and then “topped off” with the remaining components either simultaneously or sequentially. The precise manner of preparing the formulation will depend on the choice of carbonates and the percentages of the remaining components that are desirable with respect to that carbonate salt. In some embodiments, the water is in an amount between about 10-85% w/w, 15-50% w/w, or 15-45% w/w of the formulation.

The penetrant portion is a multi-component mixture, whereby the particular concentrations of the penetration enhancers are informed in part by the molecular mass of the sodium bicarbonate, or sodium bicarbonate and the therapeutic agent to be transported. The formulation enables the sodium bicarbonate and/or therapeutic agent to become bio-available to the target site within minutes of topical administration. The formulations permit the use of minimal concentrations of therapeutic agents, as little as. 1/1000th of concentrations required of alternative processes, while enabling bioactivity and positive clinical outcomes simultaneously. In some embodiments, the penetrant portion comprises an alcohol in an amount less than 5% w/w of the formulation.

One important aspect of the invention is based on the above-noted recognition that some tumors do not respond to buffer treatment as their microenvironment is not acidic and at least some of these tumors achieve metastasis by elevation of certain proteolytic enzymes that break down the extracellular matrix (ECM). If buffer treatment is contemplated, tumor cells from the biopsy of a solid tumor in a subject are therefore preferably cultured and tested in advance of treatment to insure responsiveness to buffer. Such evaluation can be carried out by any suitable means, including measurement of pH, assessment of the levels of relevant proteases, and invasion assays as impacted by buffer treatment as described in Bailey, K. M. et al (2014) supra. One important such assay is a glycolytic stress assay as described therein. Cell cultures of biopsied tumors that appear not to respond to buffer treatment as shown by such assays may benefit from administration of other antimetastatic agents and inclusion of such agents in the compositions of the invention that include buffers would also be beneficial. Thus, treatment with buffer-containing compositions alone may be contraindicated and the subject is not administered buffer as the sole active agent but diverted to alternative treatment. This does not mean, of course, that buffer is necessarily omitted from formulations used to administer alternative active agents.

The formulations comprise mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

For topical administration, and in particular transdermal administration, the formulation will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. Particularly suitable penetrants especially for those that contain at least one agent other than buffer include those that are described in the above-referenced US2009/0053290 ('290), WO2014/209910 ('910), and WO2017/127834. In addition to formulations with penetrants, transdermal delivery can be affected by mechanically disrupting the surface of the skin to encourage penetration, or simply by supplying the formulation applied to the skin under an occlusive patch.

Alternatively, the penetrant portion comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Lecithin organogel is a combination of lecithin with a gelling component, which is typically amphiphilic. Suitable gelling components also include isopropyl palmitate, ethyl laurate, ethyl myristate and isopropyl myristate. In some embodiments, the formulation comprises a gelling agent in an amount less than 5% w/w of the formulation. Certain hydrocarbons, such as cyclopentane, cyclooctane, trans-decalin, trans-pinane, n-pentane, n-hexane, n-hexadecane may also be used. Thus, an important permeation agent is a lecithin organogel, wherein the combination resulting from lecithin and the organic solvent acts as a permeation agent. In some embodiments, the penetrant portion comprises lecithin organogel, an alcohol, a surfactant, and a polar solvent. In some embodiments, the lecithin organogel is a combination of soy lecithin and isopropyl palmitate. In some embodiments, the penetrant portion comprises lecithin and isopropyl palmitate, undecane, isododecane, isopropyl stearate, or a combination thereof. In some embodiments, the formulation comprises Lipmax™ (sold by Lucas Meyer Cosmetics) in an amount between about 1-20% w/w or an equivalent 50/50 mixture of isopropyl palmitate and lecithin. Lecithin organogels are clear, thermodynamically stable, viscoelastic, and biocompatible jelly-like phases composed of hydrated phospholipids and appropriate organic liquid. An example of a suitable lecithin organogel is lecithin isopropyl palmitate, which is formed when isopropyl palmitate is used to dissolve lecithin. The ratio of lecithin to isopropyl palmitate may be 50:50. Illustrated below in the Examples is a formulation containing soy lecithin in combination with isopropyl palmitate; however, other lecithins could also be used such as egg lecithin or synthetic lecithins. Various esters of long chain fatty acids may also be included. Methods for making such lecithin organogels are well known in the art. In most embodiments, the lecithin organogel is present in the final formulation is less than about 20% w/w. In those compositions used to dissolve fat deposits, to alleviate pain from fat removal or in anhydrous compositions, the concentration of lecithin organogel may be as low as 0.5% w/w, 1% w/w, 5% w/w, 10% w/w or 20% w/w. In some embodiments, the penetrant portion comprises a mixture of xanthan gum, lecithin, sclerotium gum, pullulan, or a combination thereof in an amount less than 2% w/w, 5% w/w, or 10% w/w of the formulation. In some embodiments, the formulation comprises Siligel™ in an amount between about 1-5 w/w or 5-15% w/w, or an equivalent mixture of xanthan gum, lecithin, sclerotium gum, and pullulan. In some embodiments, the penetrant portion comprises a mixture of caprylic triglycerides and capric triglycerides in amount less than 2% w/w, 8% w/w, or 10% w/w of the formulation. In some embodiments, the formulation comprises Myritol® 312 in an amount between about 0.5-10% w/w, or an equivalent mixture of caprylic triglycerides and capric triglycerides.

In some embodiments, the penetrant portion comprises phosphatidyl choline in amount less than 12% w/w or 18% w/w of the formulation. In some embodiments, the penetrant portion comprises a phospholipid in amount less than 12% w/w or 18% w/w of the formulation. In some embodiments, the penetrant portion comprises a mixture of tridecane and undecane in amount less than 2% w/w, 5% w/w, or 8% w/w of the formulation. In some embodiments, the formulation comprises Cetiol Ultimate® in an amount less than about 2% w/w, 5% w/w, or 10% w/w, or an equivalent mixture of tridecane and undecane. In some embodiments, the penetrant portion comprises cetyl alcohol in amount less than 2% w/w, 5% w/w, or 8% w/w of the formulation. In some embodiments, the penetrant portion comprises benzyl alcohol in an amount less than about 2% w/w, 5% w/w, or 8% w/w. In some embodiments, the penetrant portion comprises stearic acid in an amount less than 2% w/w, 5% w/w, or 8% w/w of the formulation.

Lecithin organogels may be in the form of vesicles, microemulsions and micellar systems. In the form of self-assembled structures, such as vesicles or micelles, they can fuse with the lipid bilayers of the stratum corneum, thereby enhancing partitioning of encapsulated drug, as well as a disruption of the ordered bilayers structure. An example of a phospholipid-based permeation enhancement agent comprises a micro-emulsion-based organic gel defined as a semi-solid formation having an external solvent phase immobilized within the spaces available of a three-dimensional networked structure. This micro-emulsion-based organic gel in liquid phase is characterized by 1,2-diacyl-sn-glycero-3-phosphatidyl choline, and an organic solvent, which is at least one of: ethyl laureate, ethyl myristate, isopropyl myristate, isopropyl palmitate; cyclopentane, cyclooctane, trans-decalin, trans-pinane, n-pentane, n-hexane, n-hexadecane, and tripropylamine.

The lecithin organogels are formulated with an additional component to assist in the formation of micelles or vascular structures. In one approach, the organogels are formulated with a polar component such as water, glycerol, ethyleneglycol or formamide, in particular with water. In general, a nonionic detergent such as a poloxamer in aqueous solution is used to top off. Alternatively, an anhydrous composition may be obtained by using, instead of a polar component, a material such as a bile salt. When formulated with bile salts, the mi cellular nature of the composition is altered so that rather than a more or less spherical vesicular form, the vesicles become wormlike and are able to accommodate larger guest molecules, as well as penetrate the epidermis more effectively. Suitable bile salts include salts of deoxycholic acid, taurocholic acid, glycocholic acid, taurochenodeoxycholic acid, glycochenodeoxycholic acid, cholic acid and the like. Certain detergents, such as Tween® 80 or Span® 80 may be used as alternatives. The percentage of these components in the anhydrous forms of the composition is in the range of 1% w/w-15% w/w. In some embodiments, the range of bile salt content is 2%-6% w/w or 1%-3.5% w/w. In these essentially anhydrous forms, powdered or micronized nonionic detergent is used to top off, typically in amounts of 20%-60% w/w. In one approach to determine the amount of bile salt, the % is calculated by dividing the % w/w of lecithin by 10.

An additional component in the formulations of the disclosure is an alcohol. Benzyl alcohol and ethanol are illustrated in the Examples. in particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 5% w/w, 7% w/w, 10% w/w, and other intermediate weight percentages are included. Due to the aromatic group present in a permeation enhancement formulation such as benzyl alcohol, the molecule has a polar end (the alcohol end) and a non-polar end (the benzene end). This enables the agent to dissolve a wider variety of drugs and agents. The alcohol concentration is substantially lower than the concentration of the lecithin organogel in the composition.

In some embodiments, as noted above, the performance of the formulations is further improved by including a nonionic detergent and polar gelling agent or including bile salts and a powdered surfactant. In both aqueous and anhydrous forms of the composition, detergents, typically nonionic detergents are added. In general, the nonionic detergent should be present in an amount of at least 2% w/w to 60% w/w. Typically, in the compositions wherein the formulation is topped off with a polar or aqueous solution containing detergent, the amount of detergent is relatively low—e.g., 2%-25% w/w, or 5-15% w/w or 7-12% w/w. However, in compositions comprising bile salts that are essentially anhydrous and are topped-off by powdered detergent, relatively higher percentages are usually used—e.g., 20%-60% w/w.

In some embodiments, the nonionic detergent provides suitable handling properties whereby the formulations are gel-like or creams at room temperature. To exert this effect, the detergent, typically a poloxamer, is present in an amount between about 2-12% w/w, preferably between about 5-25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to I 00%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, the formulation also contains surfactant, typically, nonionic surfactant at 2-25% w/w along with a polar solvent wherein the polar solvent is present in an amount at least in molar excess of the nonionic surfactant. In these embodiments, typically, the composition comprises the above-referenced amounts of lecithin organogel and benzyl alcohol along with a carbonate salt with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like. In some embodiments, the detergent portion comprises a nonionic surfactant in an amount between about 2-25% w/w of the formulation; and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethyleneglycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about I % w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Typical components that may also be included in the formulations are fatty acids, terpenes, lipids, and cationic, and anionic detergents. In some embodiments, the formulation further comprises tranexamic acid in an amount less than 2% w/w, 5% w/w, or 10% w/w of the formulation. In some embodiments, the formulation further comprises a polar solvent in an amount less than 2% w/w, 5% w/w, 10% w/w, or 20% w/w of the formulation. In some embodiments, the formulation further comprises a humectant, an emulsifier, an emollient, or a combination thereof. In some embodiments, the formulation further comprises ethylene glycol tetraacetic acid in an amount less than about 2% w/w, 5% w/w, or 10% w/w. In some embodiments, the formulation further comprises almond oil in an amount less than about 5% w/w. In some embodiments, the formulation further comprises a mixture of thermoplastic polyurethane and polycarbonate in an amount less than about 5% w/w. In some embodiments, the formulation further comprises phosphatidylethanolamine in an amount less than about 5% w/w. In some embodiments, the formulation further comprises an inositol phosphatide in an amount less than about 5 w/w.

Other solvents and related compounds that may be used in some embodiments include acetamide and derivatives, acetone, n-alkanes (chain length between 7 and 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that may be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monolaurate, sorbitan trilaurate, sorbitan trioleate, sucrose coconut fatty ester mixtures, sucrose monolaurate, sucrose monooleate, tetradecyl N.N-dimethylamino acetate. Examples of suitable fatty acid. include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include α-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that may be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants may be included, such as vitamin C, vitamin E, proanthocyanidin and α-lipoic acid typically in concentrations of 0.1%-2.5% w/w.

In some applications, it is desirable to adjust the pH of the formulation to assist in permeation or to adjust the nature of the carbonate and/or of the target compounds in the subject. In some instances, the pH is adjusted to a level of pH 9-11 or 10-11 which can be done by providing appropriate buffers or simply adjusting the pH with base.

In some applications, in particular when the therapeutic agent includes an anesthetic, epinephrine or an alternate vasoconstrictor, such as phenylephrine or epinephrine sulfate may be included in the formulation if a stabilizing agent is present. Otherwise, the epinephrine should be administered in tandem since epinephrine is not stable at alkali pH.

In any of the anesthetic compositions, it may be desirable to administer the epinephrine in tandem with the transdermal anesthetic. Alternatively, treatment of the epinephrine with a chelator, such as the iron chelator Desferal® may stabilize the epinephrine sufficiently to include it in the transdermal formulation.

It is understood that some tumors do not respond to treatment with buffer, but apparently metastasize by virtue of elevated levels of proteases that attack the extracellular matrix surrounding the tumor. In any event, breakdown of the ECM would encourage metastasis. Therefore, an additional active agent that is optionally included in the compositions of the invention is one or more appropriate protease inhibitors. Particularly important are inhibitors of cathepsins, for example of cathepsin B, and inhibitors of matrix metalloproteinases (MMPs). These components are active alone or augment the effect of buffer for tumors that are not resistant to buffer treatment.

The formulations may include other components that act as excipients or serve purposes other than active anti-tumor effects. For example, preservatives like antioxidants e.g., ascorbic acid or α-lipoic acid and antibacterial agents may be included. Other components apart from therapeutically active ingredients and components that are the primary effectors of dermal penetration may include those provided for aesthetic purposes such as menthol or other aromatics, and components that affect the physical state of the composition such as emulsifiers, for example, Durasoft® (which is a mixture of thermoplastic polyurethane and polycarbonate). Typically, these ingredients are present in very small percentages of the compositions. It is understood that these latter ancillary agents are neither therapeutically ingredients nor are they components that are primarily responsible for penetration of the skin. The components that primarily effect skin penetration have been detailed as described above. However, some of these substances have some capability for effecting skin penetration. See, for example, Kunta, J. R. et al, J. Pharm. Sci. (1997) 86:1369-1373, describing penetration properties of menthol.

In embodiments where a bile salt is added to the combination of benzyl alcohol and lecithin organogel in lieu of topping off with an aqueous medium, micelles that would have been relatively spherical may become elongated and worm-like thus permitting superior penetration of the stratum corneum of the epidermis. The worm like formation of the micelles is particularly helpful in accommodating higher molecular weight therapeutic agents. As is known, bile salts are facial amphiphiles and include salts of taurocholic acid, glycocholic acid, taurochenodeoxycholic acid, glycochenodeoxycholic acid, cholic acid, deoxycholic acid. Detergents are also useful in lieu of bile salts and include Tween® 80 and Span® 80.

In another aspect, certain embodiments are directed to a sustained release drug delivery platform releases a therapeutic compound or compounds disclosed and made as a formulation described herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

The formulation described in this specification may also comprise more than one therapeutic compound as desired for the particular indication being treated, preferably those with complementary activities that do not adversely affect the other proteins. The formulations to be used for in vivo administration can be sterile. This can be accomplished, for instance, without limitation, by filtration through sterile filtration membranes, prior to, or following, preparation of the formulation or other methods known in the art, including without limitation, pasteurization.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can comprise, without limitation, one or more cream or lotion comprising one or more formulations described herein. In various embodiments, the kit can comprise formulation components for transdermal, topical, or subcutaneous administration, formulated to be administered as an emulsion coated patch. In all of these embodiments and others, the kits can contain one or more lotion, cream, patch, or the like in accordance with any of the foregoing, wherein each patch contains a single unit dose for administration to a subject.

Imaging components can optionally be included and the packaging also can include written or web-accessible instructions for using the formulation. A container can include, for example, a vial, bottle, patch, syringe, pre-filled syringe, tube or any of a variety of formats well known in the art for multi-dispenser packaging.

### Administration and Dosing

The formulations provided herein can be topically administered in any form. For administration a sufficient amount of the topical composition can be applied onto a desired area and surrounding skin. Also, the formulations can be applied to any skin surface, including for example, facial skin, and the skin of the hands, neck, chest and/or scalp.

In applying the formulations of the invention, the formulation itself is simply placed on the skin and spread across the surface and/or massaged to aid in penetration. The amount of formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of the formulation to the skin and prevents distortion of the formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

In an alternative to administering topically to intact skin, the surface of the skin may also be disrupted mechanically by the use of spring systems, laser powered systems, use of iontophoresis, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

It has been found, generally, that the requirements for effective penetration of the skin in the case of buffers as active agents are less restrictive than those required for alternative agents useful in preventing cancer metastasis. In addition, although for these indications delivery to the locus of the solid tumor, including melanoma, or melasma or gout is desirable, effective systemic pH alteration can be used as a way to diagnose the effectiveness of penetration when topical administration is employed.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of the formulation itself. The application of the formulation may be by simple massaging onto the skin or by use of devices such as syringes or pumps. Patches could also be used. In some cases, it is helpful to cover the area of application to prevent evaporation or loss of the formulation.

Where the application area is essentially skin, it is helpful to seal-off the area of application subsequent to supplying the formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In some embodiments, the disclosure is directed to administering a local anesthetic to a subject transdermally and a formulation which contains an effective amount of anesthetic along with 25%-70% w/w or 30%-60% w/w or 30%-40% w/w of lecithin organogel typically wherein the lecithin organogel comprises soy lecithin in combination with isopropyl palmitate or isopropyl myristate and benzyl alcohol in the range of 0.5%-20% w/w or 0.9%-2% w/w benzyl alcohol optionally including 1%-5% w/w or 2%-4% w/w menthol wherein the composition is topped off with a polar solution, typically an aqueous solution comprising 15%-50% w/w or 20%-40% w/w or 20%-30% w/w poloxamer, typically Pluronic® or alternatively may be an anhydrous composition comprising bile salts such as deoxycholic acid or sodium deoxycholate in the range of 4%-8% w/w, typically 6% w/w and the remainder of the composition powdered nonionic detergent, typically Pluronic®. The pH of the compositions is adjusted to 9-11, typically 10-11. The formulations are applied to the desired area of the skin and may be covered, for example, with Saran™ wrap for a suitable amount of time. Following the treatment, the skin can be repaired by applying a composition comprising linoleic acid.

A wide variety of therapeutic agents may be used in the formulations, including anesthetics, fat removal compounds, nutrients, nonsteroidal anti-inflammatory drugs (NSAIDs) agents for the treatment of migraine, hair growth modulators, antifungal agents, anti-viral agents, vaccine components, tissue volume enhancing compounds, anti-cellulite therapeutics, wound healing compounds, compounds useful to effect smoking cessation, agents for prevention of collagen shrinkage, wrinkle relief compounds such as Botox®, skin-lightening compounds, compounds for relief of bruising, cannabinoids including cannabidiols for the treatment of epilepsy, compounds for adipolysis, compounds for the treatment of hyperhidrosis, acne therapeutics, pigments for skin coloration for medical or cosmetic tattooing, sunscreen compounds, hormones, insulin, corn/callous removers, wart removers, and generally any therapeutic or prophylactic agent for which transdermal delivery is desired. As noted above, the delivery may simply affect transport across the skin into a localized subdermal location, such as treatment of nail fungus or modulation of hair growth or may effect systemic delivery such as is desirable in some instances where vaccines are used.

In addition to the compositions and formulations of the invention per se, the methods may employ a subsequent treatment with linoleic acid. As transdermal treatments generally open up the skin barrier, which is, indeed, their purpose, it is useful to seal the area of application after the treatment is finished. Thus, treatment with the formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow carbonates to pass through the epidermis to the dermis at least, and, if systemic administration is achieved, through the dermis itself

For administration of anesthetics as the therapeutic agent, the local anesthetic may be one or more of the following: benzocaine, lidocaine, tetracaine, bupivacaine, cocaine, etidocaine, mepivacaine, pramoxine, prilocaine, procaine, chloroprocaine, oxyprocaine, proparacaine, ropivacaine, dyclonine, dibucaine, propoxycaine, chloroxylenol, cinchocaine, dexivacaine, diamocaine, hexylcaine, levobupivacaine, propoxycaine, pyrrocaine, risocaine, rodocaine, and pharmaceutically acceptable derivatives and bioisosteres thereof. Combinations of anesthetic agents may also be used. The anesthetic agent{s) are included in the composition in effective amount(s). Depending on the anesthetic(s) the amounts of anesthetic or combination is typically in the range of 1 w/w to 50% w/w. The compositions of the invention provide rapid, penetrating relief that is long lasting. The pain to be treated can be either traumatic pain and/or inflammatory pain.

In one embodiment, the anesthetic is administered to relieve the pain associated with invasive fat deposit removal. Specific removal of fat deposits has been attractive for both health and cosmetic reasons. Among the methods employed are liposuction and injection of a cytolytic agent for fat such as deoxycholic acid (DCA). For example, a series of patents issued or licensed to Kythera Biopharmaceuticals is directed to methods and compositions for non-surgical removal of localized fat that involves injecting compositions containing DCA or a salt thereof. Representative issued patents are directed to formulation (U.S. Pat. No. 8,367,649); method-of-use (U.S. Pat. Nos. 8,846,066; 7,622,130; 7,754,230; 8,298,556); and synthetic DCA (U.S. Pat. No. 7,902,387).

In this aspect of the invention, conventional invasive fat removal techniques are employed along with administering a pain-relieving effective agent—typically lidocaine or related anesthetics via transdermal administration. In some embodiments, the pain-relieving transdermal formulation is applied to the area experiencing pain immediately before, during or immediately after the invasive fat-removal procedure.

Additional therapeutic agents may be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol may be included in an amount ranging from about 0.1% w/w to about 1.0% w/w.

The compositions containing anesthetics are useful for temporary relief of pain and itching associated with minor burns, cuts, scrapes, skin irritations, inflammation and rashes due to soaps, detergents or cosmetics, or, as noted above, pain associated with removal of fat deposits.

The benefits of alkaline pH include higher penetration capability and adjustment of the active form of the fat dissolving compound when the anesthetic is used in conjugation therewith. For example, the pKa of the deoxycholic acid is 6.58 and the pH of fat is neutral. When deoxycholic acid (DCA) is injected without buffering, it is approximately an equilibrium between the protonated and unprotonated forms. Utilizing formulations with high pH buffering shifts the balance significantly to unprotonated form making the DCA more water soluble and more likely to emulsify fats.

The formulations can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, of from 1 week to 2 weeks, for 1 week, for 2 weeks, for 3 weeks, for 4 weeks, or for 4 weeks or more. In some instances, it may also be desirable to continue treatment indefinitely for example to inhibit or prevent carcinogenesis or for improving, extending the duration of remission, or maintaining remission of a cancer or another disease or disorder. A suitable administration for a formulation comprising a skin cream, lotion or ointment, for example is once, twice, three, four times daily, or hourly if needed.

The formulations provided herein can be applied in a therapeutically effective amount. Suitable amounts, for example, per application can include, for example, from about 1 gram to about 500 grams; from about 1 gram to about 10 grams; from about 10 grams to about 25 grams; from about 10 grams to about 50 grams; from about 10 grams to about 100 grams; from about 10 grams to about 200 grams; from about 10 grams to about 350 grams; from about 10 grams to about 500 grams; from about 20 grams to about 500 grams; from about 20 grams to about 350 grams; from about 20 grams to about 200 grams; from about 20 grams to about 100 grams; from about 20 grams to about 90 grams; from about 20 grams to about 80 grams; from about 20 grams to about 70 grams; from about 20 grams to about 60 grams; from about 20 grams to about 50 grams; from about 30 grams to about 100 grams; from about 30 grams to about 80 grams; from about 30 grams to about 70 grams; or from about 30 grams to about 60 grams. Alternatively, suitable amounts, for example, per application can include, for example, at least 5 grams; at least 10 grams; at least 15 grams; at least 20 grams; at least 25 grams; at least 30 grams; at least 35 grams; at least 40 grams; at least 50 grams; at least 55 grams; at least 60 grams; at least 65 grams; at least 70 grams; at least 75 grams; at least 80 grams; at least 85 grams; at least 90 grams; at least 100 grams; or more.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A formulation in accordance with the subject matter described herein may be a topical dosage form packaged in, for example, a multi-use or single-use package, including for example, a tube, a tottle, a pump, a container or bottle, a vial, a jar, a packet, or a blister package.

Single dosage kits and packages containing a once per day amount of the topical formulation may be prepared. Single dose, unit dose, and once-daily disposable containers of the topical formulation are also provided.

The present topical formulation remains stable in storage for periods including up to about 5 years, between about 3 months and about 5 years, between about 3 months and about 4 years, between about 3 months and about 3 years, and alternately any time period between about 6 months and about 3 years.

A topical formulation described herein remains stable for up to at least 3 years at a temperature of less than or equal to 40° C. In an embodiment, the presently described topical formulation remains stable for at least 2 years at a temperature of less than or equal to 40° C. In an embodiment, the presently described formulation or emulsion remains stable for at least 3 years at a temperature of less than or equal to 40° C. and at a humidity of up to 75% RH, for at least 2 years at a temperature of less than or equal to 40° C. and at a humidity of up to 75% RH, or for at least 3 years at a temperature of less than or equal to 30° C. and at a humidity of up to 75% RH. In a further embodiment, the presently described biocompatible composition in accordance with the subject matter described herein remains stable for an extended period of time when packaged in a multi-use container such as a bottle dispenser or the like, and exhibits equal to or even greater stability when packaged in a single-use package.

In another aspect, the pharmaceutical composition of certain embodiments comprises a daily dose of a pH modulating composition or buffer (e.g. sodium bicarbonate as a topical formulation). A daily dose for topical or transdermal administration of any given pH modulating compound depends on the compound and animal and may be easily determined by the skilled artisan, a suitable amount is about 1 mg/kg to about 5 g/kg, and more typically the daily dose is about 10 mg/kg to about 5 g/kg, about 25 mg/kg to about 2000 mg/kg, about 50 mg/kg to about 2000 mg/kg, about 25 mg/kg to about 1000 mg/kg, about 50 mg/kg to about 1000 mg/kg, about 100 mg/kg to about 700 mg/kg, about 100 mg/kg to about 500 mg/kg, about 150 mg/kg to about 500 mg/kg, about 150 mg/kg to about 400 mg/kg, about 200 mg/kg to about 500 mg/kg, about 200 mg/kg to about 450 mg/kg, about 200 mg/kg to about 400 mg/kg, about 250 mg/kg to about 450 mg/kg, about 250 mg/kg to about 400 mg/kg, about 250 mg/kg to about 350 mg/kg, and about 275 mg/kg to about 325 mg/kg.

Alternatively, a suitable daily dose for topical or transdermal administration of a pH modulating composition or buffer (e.g. sodium bicarbonate) is at least about 1 mg/kg, at least about 10 mg/kg, at least about 25 mg/kg, at least about 30 mg/kg, at least about 35 mg/kg, at least about 40 mg/kg, at least about 41 mg/kg, at least about 42 mg/kg, at least about 43 mg/kg, at least about 44 mg/kg, at least about 45 mg/kg, at least about 46 mg/kg, at least about 47 mg/kg, at least about 48 mg/kg, at least about 49 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 90 mg/kg, at least about 100 mg/kg, at least about 125 mg/kg, at least about 150 mg/kg, at least about 160 mg/kg, at least about 170 mg/kg, at least about 175 mg/kg, at least about 180 mg/kg, at least about 190 mg/kg, at least about 200 mg/kg, at least about 225 mg/kg, at least about 250 mg/kg, at least about 275 mg/kg, at least about 300 mg/kg, at least about 325 mg/kg, at least about 350 mg/kg, at least about 375 mg/kg, at least about 400 mg/kg, at least about 425 mg/kg, at least about 450 mg/kg, at least about 475 mg/kg, at least about 500 mg/kg, at least about 550 mg/kg, at least about 600 mg/kg, at least about 700 mg/kg, at least about 800 mg/kg, at least about 900 mg/kg, at least about 1 g/kg, at least about 2 g/kg, at least about 3 g/kg, or at least about 5 g/kg.

Alternatively, a suitable dose for topical or transdermal administration of a pH modulating formulation or buffer (e.g. sodium bicarbonate) for subject (e.g. a human patient) is at least about 100 mg, at least about 500 mg, at least about 1 g, at least about 5 g, at least about 10 g, at least about 15 g, at least about 16 g, at least about 17 g, at least about 18 g, at least about 19 g, at least about 20 g, at least about 21 g, at least about 22 g, at least about 23 g, at least about 24 g, at least about 25 g, at least about 26 g, at least about 27 g, at least about 28 g, at least about 29 g, at least about 30 g, at least about 35 g, at least about 40 g, at least about 45 g, at least about 50 g, at least about 60 g, at least about 75 g, at least about 100 g, at least about 200 g, at least about 500 g, or at least about 1.0 kg. This does may be administered daily, twice a day, three times a day, four times a day, five times a day, or more than five times a day.

In another aspect, in certain embodiments a pH modulating composition or buffer (e.g. sodium bicarbonate) is administered topically or transdermally such that the dose results in a subject intake of at least about 0.1 nmol/hr/Kg, at least about 0.5 nmol/hr/Kg, at least about 0.7 nmol/hr/Kg, at least about 1.0 nmol/hr/Kg, at least about 1.1 nmol/hr/Kg, at least about 1.2 nmol/hr/Kg, at least about 1.3 nmol/hr/Kg, at least about 1.4 nmol/hr/Kg, at least about 1.5 nmol/hr/Kg, at least about 1.6 nmol/hr/Kg, at least about 1.7 nmol/hr/Kg, at least about 1.8 nmol/hr/Kg, at least about 1.9 nmol/hr/Kg, at least about 2.0 nmol/hr/Kg, at least about 2.5 nmol/hr/Kg, at least about 3.0 nmol/hr/Kg, at least about 3.5 nmol/hr/Kg, at least about 4.0 nmol/hr/Kg, at least about 5 nmol/hr/Kg, at least about 10 nmol/hr/Kg, at least about 25 nmol/hr/Kg, at least about 50 nmol/hr/Kg, at least about 100 nmol/hr/Kg, at least about 500 nmol/hr/Kg, or at least about 1 nmol/hr/Kg,

In another aspect, in certain embodiments a pH modulating composition or buffer (e.g. sodium bicarbonate) is administered topically or transdermally such that the dose results in a peak plasma concentration of a buffering or pH modulating compound ranges from about 1 μg/ml to 50 μg/ml, about 5 μg/ml to about 45 μg/ml, about 5 μg/ml to about 40 μg/ml, about 5 μs/ml to about 35 μg/ml, about 5 μg/ml to about 30 μg/ml, about 5 μg/ml to about 25 μg/ml, about 1 μg/ml to about 45 μg/ml, about 1 μg/ml to about 40 μs/ml, about 1 μg/ml to about 35 μg/ml, about 1 μg/ml to about 30 μg/ml, about 1 μg/ml to about 25 μg/ml, about 1 μg/ml to about 20 μg/ml, about 1 μg/ml to about 15 μg/ml, about 1 μg/ml to about 10 μg/ml, about 1 μg/ml to about 9 μg/ml, about 1 μg/ml to about 8 μg/ml, about 1 μg/ml to about 7 μg/ml, about 1 μg/ml to about 6 μg/ml, and about 1 μg/ml to about 5 μg/ml.

In another aspect, in certain embodiments a pH modulating composition or buffer (e.g. sodium bicarbonate) is administered topically or transdermally so that plasma concentration ranges from about 1 ng/ml to 500 μg/ml, about 10 ng/ml to 500 μg/ml, about 100 ng/ml to 500 μg/ml, about 1 μg/ml to 500 μg/ml, about 10 μg/ml to 500 μg/ml, about 25 μg/ml to 500 μg/ml, about 25 μg/ml to about 450 μg/ml, about 25 μg/ml to about 400 μg/ml, about 25 μg/ml to about 350 μg/ml, about 25 μg/ml to about 300 μg/ml, about 25 μg/ml to about 250 μg/ml, about 50 μg/ml to about 500 μg/ml, about 55 μg/ml to about 500 μg/ml, about 60 μg/ml to about 500 μg/ml, about 65 μg/ml to about 500 μg/ml, about 70 μg/ml to about 500 μg/ml, about 75 μg/ml to about 500 μg/ml, about 80 μg/ml to about 500 μg/ml, about 85 μg/ml to about 500 μg/ml, about 90 μg/ml to about 500 μg/ml, about 95 μg/ml to about 500 μg/ml, about 100 μg/ml to about 500 μg/ml, about 110 μg/ml to about 500 μg/ml, about 120 μg/ml to about 500 μg/ml, about 130 μg/ml to about 500 μg/ml, about 140 μg/ml to about 500 μg/ml about 150 μg/ml to about 500 μg/ml, about 160 μg/ml to about 500 μg/ml, about 170 μg/ml to about 500 μg/ml, about 180 μg/ml to about 500 μg/ml, about 200 μg/ml to about 500 μg/ml, about 200 μg/ml to about 490 μg/ml, about 200 μg/ml to about 480 μg/ml, about 200 μg/ml to about 470 μg/ml, about 200 μg/ml to about 460 μg/ml, about 200 μg/ml to about 450 μg/ml, about 200 μg/ml to about 440 μg/ml, about 200 μg/ml to about 430 μg/ml, or about 200 μg/ml to about 400 μg/ml.

In further embodiments, a pH modulating composition or buffer (e.g. sodium bicarbonate) is administered topically or transdermally so that plasma concentration is at least 10 ng/ml, at least 25 ng/ml, at least 50 ng/ml, at least 100 ng/ml, at least 250 ng/ml, at least 0.5 μg/ml, at least 0.75 μg/ml, at least 1 μg/ml, at least 2 μg/ml, at least 3 μg/ml, at least 4 μg/ml, at least 5 μg/ml, at least 6 μg/ml, at least 7 μg/ml, at least 8 μg/ml, at least 9 μg/ml, at least 10 μg/ml, at least 15 μg/ml, at least 20 μg/ml, at least 25 μg/ml, at least 30 μg/ml, at least 35 μg/ml, at least 40 μg/ml, at least 45 μg/ml, at least 50 μg/ml, at least 55 μg/ml, at least 60 μg/ml, at least 65 μg/ml, at least 70 μg/ml, at least 75 μg/ml, at least 80 μg/ml, at least 85 μg/ml, at least 90 μg/ml, at least 95 μg/ml, at least 100 μg/ml or more than 100 μg/ml.

In another aspect, a pH modulating compound or buffer (e.g. sodium bicarbonate) is administered topically or transdermally so that peak plasma concentration is reached in 10 min, 15 min, 20 min, 30 min, 45 min, 60 min, 75 min, 90 min, 2 hr, 3 hr, 4 hr, 5 hr, 6 hr, 7 hr, 8 hr, 10 hr, 12 hr or 24 hr after administration.

Aspects of the present specification disclose that the symptoms associated with a disease or disorder described herein are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

### Coadministration w Anti-Cancer and Immunotherapy Agents

In another aspect, formulations and/or compounds provided herein are coadministered or administered to an animal, subject or patient in conjunction with one or more chemotherapeutic compounds such as alkylating agents, antibodies and related agents with anti-tumor properties, anthracyclines, antimetabolites, antitumor antibiotics, aromatase inhibitors, cytoskeletal disruptors (e.g. taxanes), epothilones, histone deacetylace inhibitors, kinase inhibitors, nucleoside analogues, topoisomerase inhibitors, retinoids, vinca alkaloids and derivatives, and the like. The administration or co-administration of one or more formulation or composition of the invention and one or more chemotherapeutic agents can be used for the treatment of tumors or cancer in an animal, subject or patient.

As an example, alkylating agents can be administered or coadministered with or as part of a formulation provided herein. Examples of an alkylating agents that can be co-administered include mechlorethamine, chlorambucil, ifosfamide, melphalan, busulfan, carmustine, lomustine, procarbazine, dacardazine, cisplatin, carboplatin, mitomycin C, cyclophosphamide, ifosfamide, thiotepa, and dacarbazine, and analogues thereof. See for example U.S. Pat. No. 3,046,301 describing the synthesis of chlorambucil, U.S. Pat. No. 3,732,340 describing the synthesis of ifosfamide, U.S. Pat. No. 3,018,302 for the synthesis of cyclophosphamide, U.S. Pat. No. 3,032,584 describing the synthesis of melphalan, and Braunwald et al., “Harrison's Principles of Internal Medicine,” 15th Ed., McGraw-Hill, New York, N.Y., pp. 536-544 (2001) for clinical aspects of cyclophosphamide, chlorambucil, melphalan, ifosfamide, procarbazine, hexamethylmelamine, cisplatin, and carboplatin. Examples of nucleoside analogues, include, but are not limited to, fludarabine pentostatin, methotrexate, fluorouracil, fluorodeoxyuridine, CB3717, azacitidine, cytarabine, floxuridine, mercaptopurine, 6-thioguanine, cladribine, and analogues thereof.

In another aspect, formulations provided herein are administered with chemosensitising agents such as those described for example in U.S. Pat. No. 3,923,785 describing the synthesis of pentostatin, U.S. Pat. No. 4,080,325 describing the synthesis of methotrexate, U.S. Pat. No. 2,802,005 describing the synthesis of fluorouracil, and Braunwald et al., “Harrison's Principles of Internal Medicine,” 15th Ed., McGraw-Hill, New York, N.Y., pp. 536-544 (2001) for clinical aspects of methotrexate, 5-fluorouracil, cytosine arabinoside, 6-mercaptopurine, 6-thioguanine, and fludarabine phosphate. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992), incorporated by reference herein.

In another aspect, formulations provided herein can be administered or co-administered with diterpene compounds, including but not limited to paclitaxel, docetaxel, cabazitaxel, and the like.

In another aspect, formulations provided herein can be administered or co-administered with compounds that inhibit topoisomerase II or compounds that otherwise interact with nucleic acids in cells. Such compounds include, for example, doxorubicin, epirubicin, etoposide, teniposide, mitoxantrone, and analogues thereof. In one example, this combination is used in treatment to reduce tumor cell contamination of peripheral blood progenitor cells (PBSC) in conjunction with high-dose chemotherapy and autologous stem cell support (HDC-ASCT). See U.S. Pat. No. 6,586,428 to Geroni et al.

In another aspect, formulations provided herein can be administered or co-administered with immunotherapeutic agents. Immunotherapy has become a promising approach to treat cancer. Kruger C., et al., Immune based therapies in cancer, Histol. Histopathol, 2007, v22, 687-696. The types of immunotherapies used to treat cancer and can be categorized as active, passive or hybrid (active and passive). Active immunotherapy directs the immune system to attack tumor cells by targeting TAAs. Passive immunotherapies enhance existing anti-tumor responses and include the use of checkpoint inhibitors, monoclonal antibodies, lymphocytes and cytokines. A suitable immunotherapeutic agent or immunotherapy may be a biologic or biologically active agent such as an antibody or modified antibody or cell based therapy such as chimeric antigen receptor therapy (CAR-T). It is recognized that there may be overlap in categorizing and classifying such agent as biological agents, immunotherapeutic agents, cell-based therapeutics, biological therapeutic agents and the like. Examples of approved antibody immunotherapeutics include, alemtuzumab, atezolizumab, avelumab, ipilimumab, durvalumab, nivolumab, ofatumumab, rituximab, and trastuzumab. These and others are suitable for use in certain embodiments provided herein.

In another aspect, formulations can be administered or co-administered with biological therapeutic agents and other therapeutic drugs. For example, virulizin (Lorus Therapeutics), which is believed to stimulate the release of tumor necrosis factor, TNF-α, by tumor cells in vitro and stimulate activation of macrophage cells. This can be used in combination with one or more formulation of the invention to increase cancer cell apoptosis and treat various types of cancers including pancreatic cancer, malignant melanoma, kaposi's sarcoma (KS), lung cancer, breast cancer, uterine, ovarian and cervical cancer. Another example is CpG 7909 (Coley Pharmaceutical Group), which is believed to activate NK cells and monocytes and enhance ADCC. Cytokines such as interferons and interleukins (e.g. EPO, thrombopoietin) are biological agents useful certain embodiments in combination with one or more formulation of the invention. Other types of suitable biological therapeutic agents include RNA and protein bases-agents such as enzymes. These therapeutic agents and others can also be used in combination with formulations provided herein.

Another example of a biological therapeutic agent that is used for the treatment of certain cancers in certain embodiments are angiogensis inhibitors. Accordingly, formulations of the invention can also be combined with angiogensis inhibitors to increase anti-tumor effects. Angiogenisis is the growth of new blood vessels. This process allows tumors to grow and metastasize. Inhibiting angiogeneisis can help prevent metastasis, and stop the spread of tumors cells. Angiogenisis inhibitors include, but are not limited to, angiostatin, endostatin, thrombospondin, platelet factor 4, Cartilage-derived inhibitor (CDI), retinoids, Interleukin-12, tissue inhibitor of metalloproteinase 1, 2 and 3 (TIMP-1, TIMP-2, and TIMP-3) and proteins that block the angiogensis signaling cascade, such as anti-VEGF (Vascular Endothelial Growth Factor) and IFN-alpha. Angiogenesis inhibitors can be administered or co-administered with tumor specific constructs, including antigen-binding constructs capable of mediating, for example, ADCC and/or complement fixation or chemotherapy-conjugated antigen-binding of the invention to combat various types of cancers, for example, solid tumor cancers such as lung and breast cancer. Other examples of biological therapeutic agents include inhibitors of E-cadherin and of epidermal growth factor receptor (EGFR). Known inhibitors include erlotinib, an anti-integrin drug (Cilengitide), Cariporide, Eniporide and Amiloride.

In another aspect, formulations of the invention can be administered or co-administered with disease modifying anti-rheumatic agents (DMAR agents) for the treatment of rheumatoid arthritis, psoriasis, ulcerative colitus, systemic lupus erythematosus (SLE), Crohn's disease, ankylosing spondylitis, and various inflammatory disease processes. In such treatment, the constructs, for example, antigen-binding constructs, of the invention are commonly administered in conjunction with compounds such as azathioprine, cyclosporin, gold, hydroxychloroquine, methotrexate, penicallamine, sulphasalazine, and the like.

In another aspect, formulations provided herein can be used with palliative (non-radical) operations to surgically remove tumors. In this aspect, one or more formulations of the invention can be administered before and after surgical extractions of tumors in order to reduce the likelihood of metastasis and reoccurrence by killing any cancer cells that were not removed during the surgery.

Other diseases, conditions, and disorders described herein can be treated with formulations and methods provided herein.

### EXPERIMENTAL EXAMPLES

The compositions and methods described herein will be further understood by reference to the following examples, which are intended to be purely exemplary. The compositions and methods described herein are not limited in scope by the exemplified embodiments, which are intended as illustrations of single aspects only. Any methods that are functionally equivalent are within the scope of the invention. Various modifications of the compositions and methods described herein in addition to those expressly described herein will become apparent to those skilled in the art from the foregoing description and accompanying figures. Such modifications fall within the scope of the invention.

The following examples are intended to illustrate but not to limit the invention.

### Example 1—Biomechanical and Biochemical Experiments

With regard to reverse wormlike micellar systems comprised of mixtures of bile salt and lecithin in organic liquids, the chemical mixture was dissolved in methanol to form 200 mM and 100 mM stock solutions, respectively. The final samples with desired concentrations were obtained by adding organic solvent, followed by stirring till the solution became transparent and homogeneous. The studies revealed the presence of residual water at a 0.9:1 molar ratio in these samples.

Steady and dynamic rheological experiments were performed on a Rheometrics RDA-III strain-controlled rheometer. Frequency spectra were conducted in the linear viscoelastic regime of the samples, as determined from dynamic strain sweep measurements. Small angle neutron scattering (SANS) measurements were made on the NG-7 (30 m) beamline at NIST in Gaithersberg, Md. Neutrons with a wavelength of 6 A were selected. Lecithin-bile salt samples were prepared with deuterated cyclohexane and studied in 1 mm quartz cells at 25° C. The scattering spectra were corrected and placed on an absolute scale using calibration standards provided by NIST.

For dilute solutions of non-interacting scatters, the SANS intensity can be modeled purely in terms of the form factor P(q) of the scatterers. In this study, we considered form factor models for three different micellar shapes; ellipsoids, rigid cylinders and flexible cylinders. The models were implemented using software modules supplied by NIST.

### Example 2—Clinical Studies of Chemical Permeation Enhancement

Clinical trials of 200 subjects were performed as CPE compositions were applied twice daily for 45 days. Several dermatologists and plastic surgeons observed the patients. Documentation of objective results was performed with the microrelief technique. The technique relies upon the application of a polyvinylsiloxane impression material to the skin. Upon drying, the film is removed and either sputter coated with a conducting metal for visualization utilizing a scanning electronmicroscope and/or a high power stereomicroscope and photography. Each scale division equals 0.5 mm.

Three subjects, ages 45, 58 and 70 were selected. An adjacent site, which remained untreated, was used as a control. Two dermatologists who performed the biopsies were blind as to which was the treated and which was the control site.

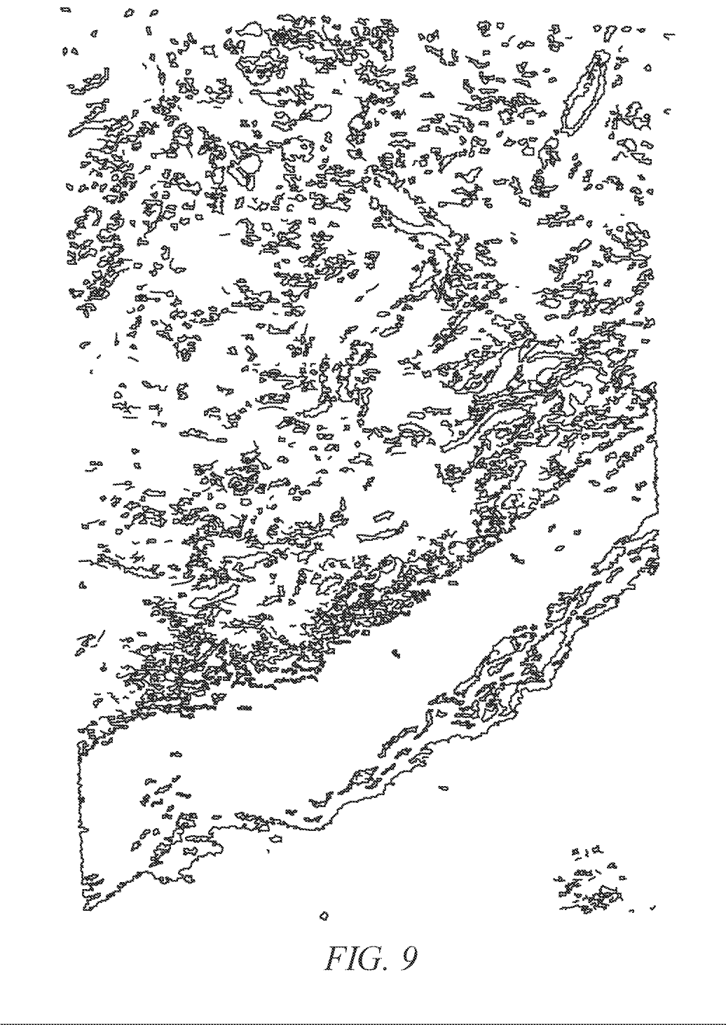

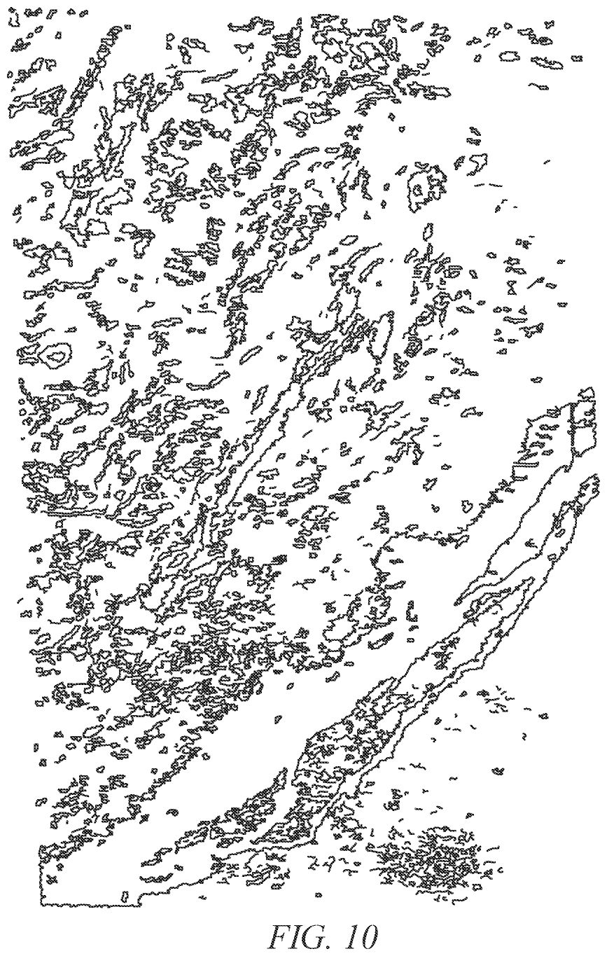

The specimens were processed for histological evaluation. Standard dehydrating and paraffin embedding procedures were used. The specimens were stained with H & E and alician blue to visualize the collagen and proteoglycan components of the extracellular matrix. Representative histological findings are demonstrated in FIGS. 9 & 10. It was clear that the treated skin showed significant differences as compared with the control. The dermis in the treated specimen shows a greater abundance of collagen with characteristics that depict a more recently deposited fibrous network. The epithelial layer is much thicker, well organized and reflects a greater cellular metabolic activity. The result confirms effective and expeditious percutaneous absorption of the guest molecules.

### Example 3—Percutaneous Penetration

This skin model utilizes normal, human-derived epidermal keratinocytes and normal, human-derived dermal fibroblasts, which have been cultured to create a multi-layered, highly differentiated model of human dermis and epidermis in a three-dimensional tissue construct, which is metabolically and mitotically active. The tissues are cultured on specially prepared cell culture inserts using serum-free medium. Ultrastructurally, this model closely parallels human skin, thus providing a useful in vivo means to assess percutaneous absorption or permeability. The model has an in vivo-like lipid profile with in vivo-like ceramides present. Furthermore, this model reproduces many of the barrier function properties of normal human skin and has been determined to be a useful substrate for percutaneous absorption, transdermal drug delivery and other studies related to the barrier function of the human.

Donor solution (PBS) containing four different concentrations (0.25 g/ml, 0.5 g/ml, 1 g/ml, and 2 g/ml) of the sample composition or control base was prepared. Neutral red (0.001%) was added to give a red tinge to the donor solution.

The donor solution was then added to the center core of the permeation device containing the skin tissue and the whole assembly was then placed into the wells of a 6 well plate containing 3 ml of PBS. At definite intervals, the assembly was moved to a fresh well containing 3 ml. of PBS. After incubation, PBS from the 6 wells were collected in separate tubes, labeled and stored in −70° C. for further processing. After 120 hrs. of incubation confirmed that all skin tissue samples in this study were viable at the end of the study period.

### Example 4—Transepidermal Water Loss Measurements

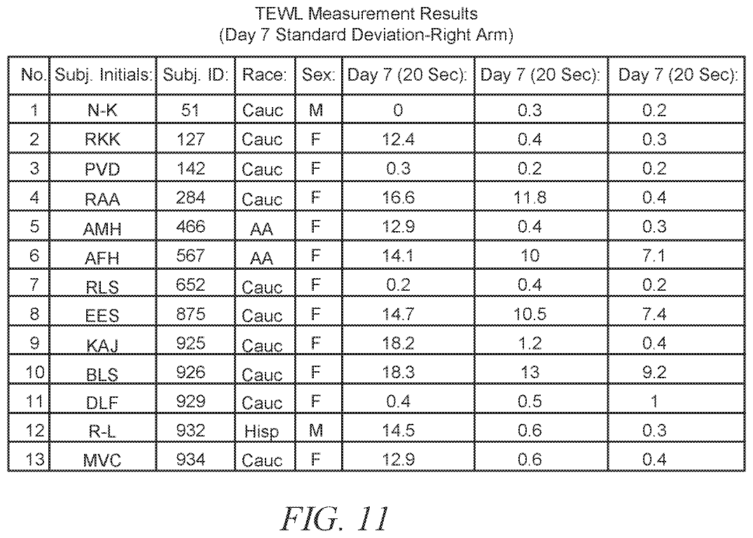

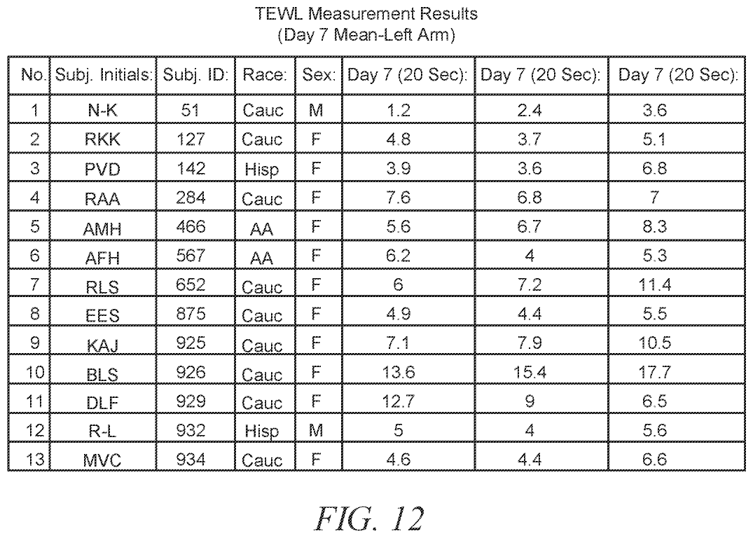

The rate of transepidermal water loss (TEWL) (g/h/m2) is reflective of the skin's barrier function. A TEWL probe utilizing the Dermalab Evaporimetry Systen (Cortex Technology, Hadsund Denmark) was used to take three baseline measurements on both the left and right volar forearms. The template demarcated test sites were then tape stripped (Duct tape, 3M, St. Paul, Minn.). Following tape stripping, TEWL measurements were again taken at each tape stripped site. Increased TEWL indicates a disruption of the permeation barrier of the SC following the topical application of the chemical permeation enhancement compositions (FIGS. 11 & 12).

### Example 5—Collagen Message Levels

A real time polymerase chain reaction method was used to determine collagen message levels in the human dermal fibroblast cell lines exposed to the penetration sample compound (at concentrations of 0.25 mg/ml) and base control (at 0.25 mg/ml concentrations) Cells incubated in media alone served as negative controls.

Absolute quantities of collagen were determined in the fibroblasts using a real time polymerase chain reaction analysis. cDNA was prepared from the fibroblasts using a retroscript real time polymerase chain reaction kit.

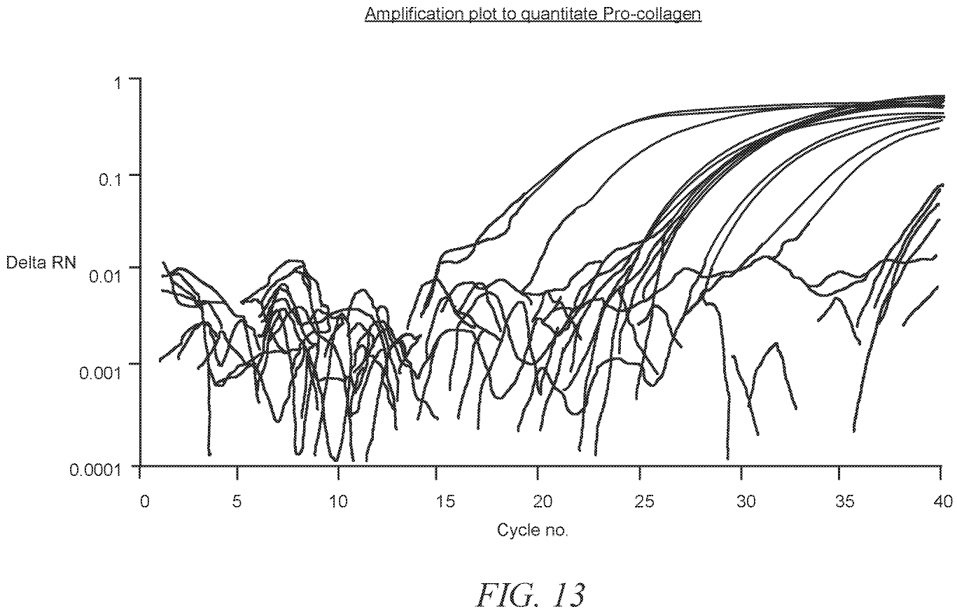

These analyses showed that exposure to the penetration sample compound induced the expression of collagen in human dermal fibroblasts within 30 minutes (FIG. 13). Similar changes were not observed at 30 minutes when the base was applied to fibroblast cultures.

These findings thus correlate with the penetration data and clearly suggest that the penetration sample compound after permeating the epidermal layer of the skin can induce collagen synthesis in human dermal fibroblast cells.

### Example 6—Electrometric Analysis of Permeability of Human Epidermis

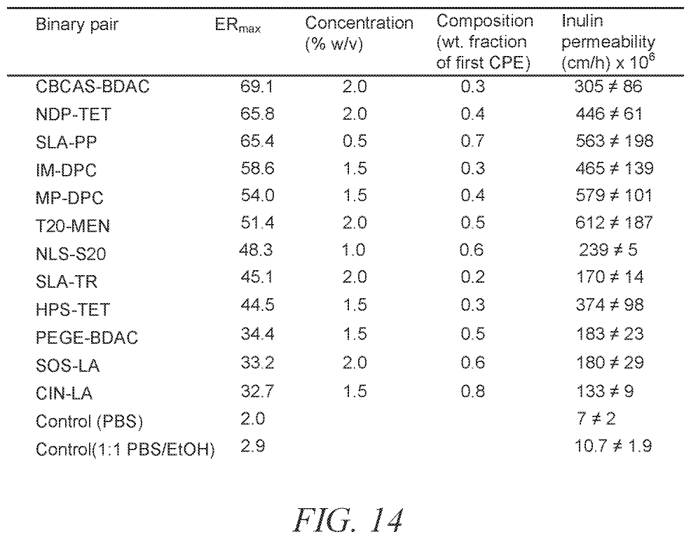

Skin conductivity is generally a good measure of its permeability to polar solutes. Transepidermal current is mediated by the movement of charge carrying ions and is thus related to the permeability of these ions. For screening purposes, the skin possessing higher electrical conductivity exhibits higher permeability to polar solutes. Therefore, monitoring electrical conductivity of skin exposed to various permeation enhancing formulations will identify the most efficient formulations in increasing skin permeability. The preferred binary mixtures of chemical permeation enhancers are illustrated in FIG. 14.

### Example 7—Elemental Analysis

A proton-induced X-ray spectrographic technique is used for the non-destructive, simultaneous elemental analysis of solid, liquid or aerosol filter samples. To determine if the sample has penetrated through the epidermal layer, the PBS samples collected after incubation were subjected to elemental analysis (Table: Elemental Analysis).

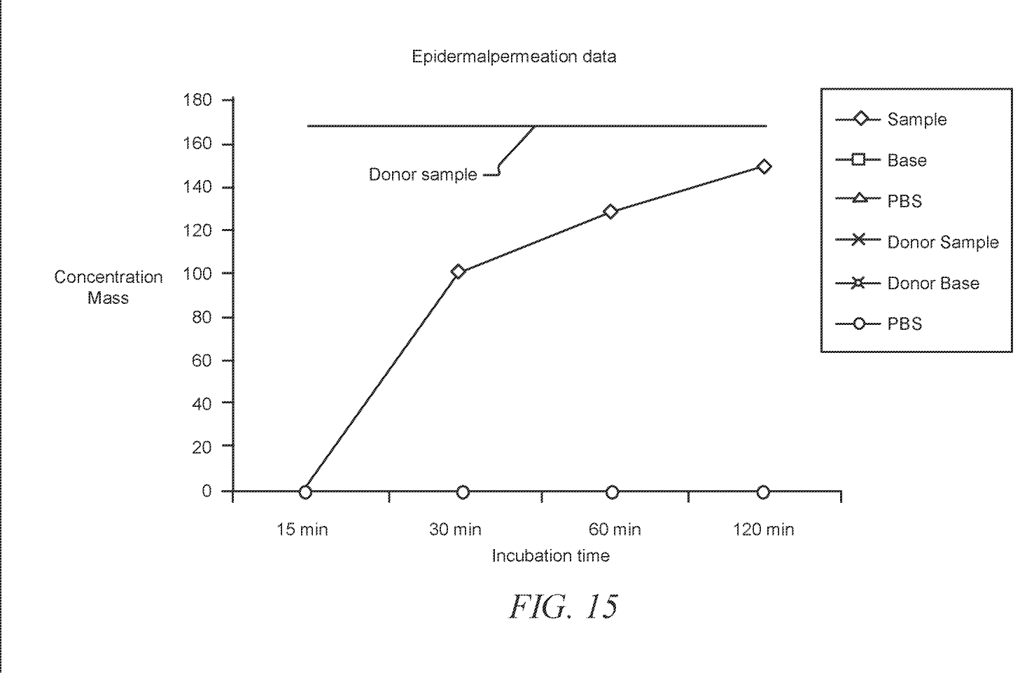

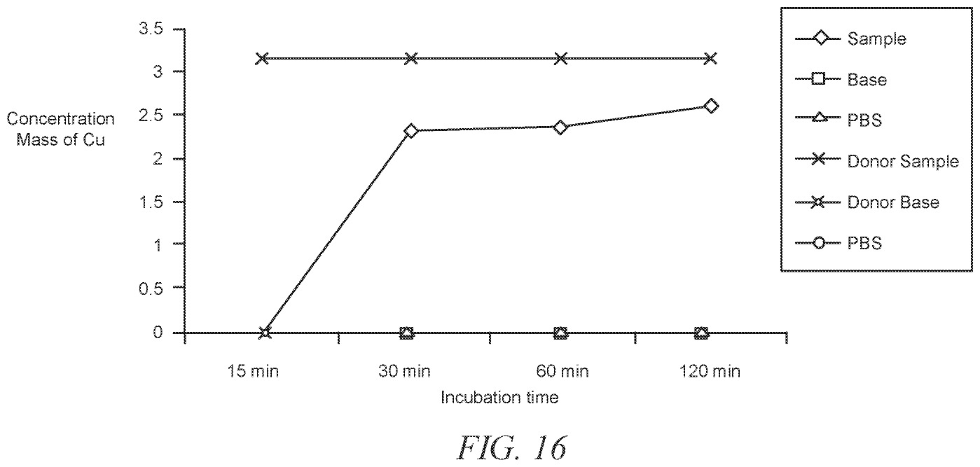

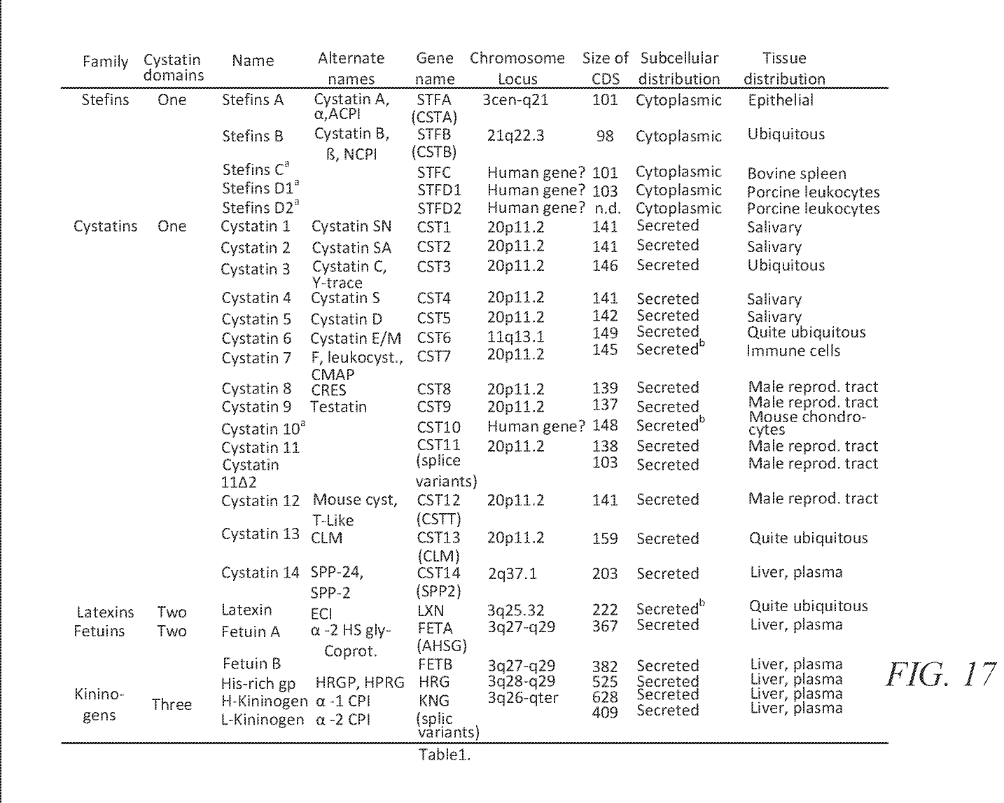

This was outsourced to Elemental Analysis Inc., Lexington, Ky. Samples were analyzed by proton induced X-ray analyzer, which measured 74 elements in one run with special interest in two elements, copper (Cu) and iron (Fe). The results are presented in FIGS. 15 & 16.

Results of the proton induced X-ray analysis confirmed that (1) the penetrant sample dose penetrated the epidermis (2) within 30 minutes of application. Thus the compound is available to the deeper layers, especially dermal fibroblasts within 30 minutes of its application to the epidermal surface.

### Example 8—High Performance Liquid Chromatography Analysis

The concentration of insulin in the receiver well at different time intervals were measured using a HPLC system. A 40:60 (v/v) mixture of acetonitrile and water was the mobile phase. Flow rate was 1.0 mL/min. and the eluent was monitored at 276 nm. linearity for HPLC analysis was observed in the concentration range of 0.01-12.5 IU/ml (R²>0.99).

This is a technique used to separate, identify, and quantify each component in a mixture. Each component in the sample interacts slightly differently with the absorbent material, causing different flow rates for the different components and leading to the separation of the components as they flow out the column. It is a mass transfer process involving adsorption.

### Example 9—Permeability Coefficient and Enhancement Factor Calculations

In this study, the amount of drug permeated was calculated as the total amount of drug permeated through skin during a time period of 48 hours. The lag time were calculated as the x-intersept of the steady state portion of the permeation profiles (cumulative insulin permeated, IU/cm²) plotted against the time (hr) profiles.

The following steady-state equation was used to calculate permeability of the skin:

Amount of drug permeated=Aₘ*C₀*Kₚ*t

where, Aₘ is the exposure area of the skin sample (0.64 cm²), C₀ is the initial concentration in the well in mM, Kₚ is the permeability of the membrane and t is time in hrs. The permeability is give in terms of the diffusion coefficient (Dₘ), the partition coefficient (Kₘ), and the thickness of the skin sample (L):

Kₚ=DₘKₘ/L a.

### Example 10—Use of Topical Buffering Agents to Decrease Primary Tumor Metastases and Increase Survival in Metastatic Breast Cancer

In this experiment, the small molecule protease inhibitor JPM-OEt in formulations of the invention was tested for its ability to inhibit multiple stages of tumor progression with and without coadministration and coformulation of topically applied buffering agents in formulations of the invention.

In vivo tests were performed as follows: RIP1-Tag2 mice were treated with topically applied JPM-OEt with and without topically applied buffering formulations. Trial design was built to assess these primary outcomes: the effects on angiogenesis and tumor growth. Treatment groups randomized as follows:

a. Control Group

b. Treatment Group 1: 100 μL of JPM-OEt formulation applied once daily, formulation detailed below

c. Treatment Group 2: 100 μL of JPM-OEt formulation, once absorbed, followed by 100 μL of buffering formulation. Each applied topically once daily. Formulations are detailed below:

TABLE 1
JPM-OEt Formulation

| Ingredient | % Weight |
|---|---|
| Menthol | 0.5% |
| Benzyl Alcohol | 1.0% |
| LIP | 25.0% |
| Cetyl Alcohol | 1.5% |
| Stearic Acid | 1.5% |
| Deionized Water | 31% |
| Ethanol | 1.5% |
| 30% Pluronic Gel | 35.0% |
| JPM-OEt | 2% |
| Durosoft PK-SG | 1.0% |
| Total | 100.0% |

TABLE 2
Buffering Formulation

| Ingredient | % weight |
|---|---|
| Menthol | 0.50% |
| Ethanol | 1.50% |
| Benzyl Alcohol | 1.00% |
| Cetyl Alcohol | 2.00% |
| Almond Oil | 3.00% |
| LIP | 14.00% |
| Propylene Glycol | 5.00% |
| 30% Pluronic Gel | 18.00% |
| DI Water | 21.00% |
| Sodium Bicarbonate (65 μm) | 33.00% |
| Durosoft PK-SG | 1.00% |
| Total | 100.00% |

Daily application of both treatment groups resulted in significant reductions in angiogenic activity. Treatment Group 1, JPM-OEt alone, produced a 49% reduction in the number of angiogenic islets evident at 10.5 weeks of age relative to the Control Group. Treatment Group 2, JPM-OEt coadministered with a topically buffering agent, resulted in a 66% reduction in the number of angiogenic islets evident at 10.5 weeks of age relative to the Control Group.

Daily application of both treatment groups resulted in significant reductions in tumor growth rates. As previously observed buffering activity did not significantly increase the effectiveness along this end point. Treatment Group 1, JPM-OEt alone, observed a 67% reduction of cumulative tumor volume at 14.5 weeks of age relative to the Control Group. Treatment Group 2 observed a similar reduction of 70% of cumulative tumor volume at 14.5 weeks of age relative to the Control Group.

### Example 11—Use of Topical Agents to Deliver JM-565 to Inhibit Tumor Growth in the MMTV-PyMT Mouse Breast Cancer Model

In this experiment, the small molecule JPM-565 in formulations of the invention was tested for its ability to inhibit tumor growth.

In vivo tests were performed as follows: FVB/N mice were inoculated with 5×10⁵ primary MMTV-PyMT tumor cells in the mammary gland of congenic immunocompetent recipient mice. Once tumor volume reached 125 mm³, mice were treated with a topically applied JPM-565 formulation 3 times daily. The experiment was ended at Day 21. At the end of treatment, tumors were excised and their volumes determined. The anti-tumor effect was compared to controls. Treatment groups were randomized as follows:

Control Group: No treatment

Treatment Group: 2004 of JPM-OEt formulation applied three times daily, formulation detailed below:

TABLE 3
Treatment Group Formulation

| Ingredient | % Weight |
|---|---|
| Menthol | 0.5% |
| Benzyl Alcohol | 1.0% |
| LIP | 25.0% |
| Cetyl Alcohol | 1.5% |
| Stearic Acid | 1.5% |
| Deionized Water | 10.0% |
| Ethanol | 1.5% |
| 30% Pluronic Gel | 25.0% |
| NaOH 50% Solution | 1.0% |
| Sodium Bicarbonate (100 μm) | 30.0% |
| JPM-565 | 2.0% |
| Durosoft PK-SG | 1.0% |
| Total | 100.0% |

Topical application of JPM-565 in the Treatment Group displayed a 45% reduction in tumor growth compared to control at Day 21 when the experiment was ended. Further, cell proliferation was quantified by immunohistochemical detection of the proliferation marker Ki67, revealing a significant decrease in the proliferation rate of tumors in the JPM-565 Treatment Group compared to the Control Group.

### Example 12—Use of Topical E-64 to Decrease Primary Tumor Metastases and Increase Survival in Buffer Resistant Cell Line

In this experiment, topical applications of E-64 with formulations of the invention were tested for their ability to influence the tumor microenvironment and inhibit the spread of metastases and increase overall survival in a mouse model for lung carcinoma. The topical formulations of the invention were compared to a negative and positive controls as detailed below.

In vivo tests were performed as follows: SCID-beige mice aged 6 weeks were injected intravenously with 1×10⁶ LL/2 cells. The following day after tumor inoculation, mice were then randomized into 3 treatment groups as outlined below.

The treatment groups were:

Group A (Negative Control): Untreated

Group B (Positive Control): 200 mM sodium bicarbonate drinking water ad libitum

Group C: 100 μL×3 doses daily (total daily dose of 300 μL) of formulation detailed below

The formulation of the invention was as follows:

TABLE 4
Group C Formulation

| Ingredient | % Weight |
|---|---|
| Menthol | 0.5% |
| Benzyl Alcohol | 1.0% |
| Cetyl Alcohol | 2.0% |
| Stearic Acid | 2.0% |
| Almond Oil | 3.0% |
| LIP | 14.0% |
| Ethanol | 1.5% |
| Propylene Glycol | 5.0% |
| 30% Pluronic Gel | 18.0% |
| DI Water | 19.0% |
| 50% NaOH Solution | 1.0% |
| Sodium Bicarbonate (100 μm) | 30.0% |
| E-64 | 2.0% |
| Durosoft PK-SG | 1.0% |
| Total | 100.0% |

Application of transdermal agent in treatment group C occurred 3 times/day for 120 days. Volumes of primary tumors were measured twice weekly. Mice were euthanized by cervical dislocation when tumor burden became excessive (primary, intraperitoneal, or lymph node >2000 mm³) or when mouse progressed to a moribund state. Survival data were expressed as a Kaplan-Meier curve.

Upon termination of the survival experiment, tumor metastases were identified by gross necropsy. All tumor tissue was fixed in 10% neutral buffered formalin (NBF). The green fluorescent (GFP) tumors were detected using a 470 nm/40 nm excitation filter and imaged using a mounted digital camera. Whole lung images data were analyzed with Adobe Photoshop 5.0 using the “magic wand” tool to select lung area and green fluorescent tumor lesions. Pixel area of the selected images was measured using ImageJ.

There were significant differences observed in survival. Topically applied E-64 demonstrated better survival compared to both the negative and positive control groups, as follows:

of mice surviving to 120 days: Group A: 20%; Group B: 20%; Group C: 45%

### Example 13—Synergistic Use of Topical JPM-OEt Coformulated with Topical Buffering Agents to Decrease Primary Tumor Metastases and Increase Survival in Metastatic Breast Cancer

In this experiment, topical applications of JPM-OEt, combined with buffer, in formulations of the invention were tested for their ability to synergistically influence the tumor microenvironment to inhibit the spread of metastases and increase overall survival in a mouse model for metastatic breast cancer. The topical formulations of the invention were compared to a “no treatment” control as well as orally delivered buffer as a positive control.

In vivo tests were performed as follows: 72 female Ncr nude mice aged 6 weeks were injected with 5×10⁶ MDA-MB-231/eGFP cells in the mammary fatpad to generate orthotopic “primary” tumors. The following day after tumor inoculation, mice were then randomized into 5 treatment groups as outlined below.

The treatment groups were:

Group A: Untreated Control

Group B: 200 mM sodium bicarbonate drinking water ad libitum

Group C: 50 μL×3 doses daily (total daily dose of 150 μL) of formulation detailed below (Buffer alone)

Group D: 100 μL×3 doses daily (total daily dose of 300 μL) of formulation detailed below (JPM-OEt alone)

Group E: 150 μL×3 doses daily (total daily dose of 450 μL) of formulation detailed below (JPM-OEt and Buffer together)

The formulations of the invention are shown in Table 5 were as follows:

TABLE 5

| Group | C (Buffer Alone) |
|---|---|
| Ingredient | % weight |
| Menthol | 0.50% |
| Benzyl Alcohol | 1.00% |
| LIP | 25.00% |
| Cetyl Alcohol | 1.50% |
| Stearic Acid | 1.50% |
| Deionized Water | Q.S. |
| Ethanol | 1.50% |
| 30% Pluronic Gel | 30.56% |
| NaOH 50% Solution | 1.00% |
| Sodium Bicarbonate | 33.20% |
| Durosoft PK-SG | 1.00% |
| Total | 100.00% |
| Group | D (JPM-OEt Alone) |
| Ingredient | % weight |
| Menthol | 0.50% |
| Benzyl Alcohol | 1.00% |
| LIP | 25.00% |
| Cetyl Alcohol | 1.50% |
| Stearic Acid | 1.50% |
| Deionized Water | Q.S. |
| Ethanol | 1.50% |
| 30% Pluronic Gel | 35.00% |
| NaOH 50% Solution | 1.00% |
| JPM—OEt | 2.00% |
| Durosoft PK-SG | 1.00% |
| Total | 100.00% |
| Group | E (JPM-OEt + Buffer) |
| Ingredient | % weight |
| Menthol | 0.50% |
| Benzyl Alcohol | 1.00% |
| LIP | 25.00% |
| Cetyl Alcohol | 1.50% |
| Stearic Acid | 1.50% |
| Deionized Water | Q.S. |
| Ethanol | 1.50% |
| 30% Pluronic Gel | 30.56% |
| NaOH 50% Solution | 1.00% |
| JPM—OEt | 2.00% |
| Sodium Bicarbonate | 33.20% |
| Durosoft PK-SG | 1.00% |
| Total | 100.00% |

Application of transdermal agent in treatment groups C, D, and E occurred 3 times/day for 120 days. Volumes of primary tumors in mammary fat pads were measured twice weekly and calculated from orthogonal measurements of external dimensions as (width)²×(length)/2. Surgical resections of primary tumors occurred when tumors reached 350-500 mm³. Mice were euthanized by cervical dislocation when tumor burden became excessive (primary, intraperitoneal, or lymph node >2000 mm³) or when mouse progressed to a moribund state. Survival data were expressed as a Kaplan-Meier curve.

Upon termination of the survival experiment, tumor metastases were identified by gross necropsy. All tumor tissue was fixed in 10% neutral buffered formalin (NBF). The green fluorescent (GFP) tumors were detected using a 470 nm/40 nm excitation filter and imaged using a mounted digital camera. Whole lung images data were analyzed with Adobe Photoshop 5.0 using the “magic wand” tool to select lung area and green fluorescent tumor lesions. Pixel area of the selected images was measured using ImageJ.

The primary tumor growth rates were observed to be the same in all active Groups, Groups B, C, D, and E. This is consistent with earlier findings.

There were significant differences observed in metastatic rates in all treatment groups, Groups B, C, D, and E. As follows:

In the Untreated Group a (No Treatment, Negative Control), Metastatic Rates were as Follows:

A. Intestinal: 36%

B. Mesentery: 14%

C. Lymph Node: 64%

D. Lung: 79%

In the Orally Treated, Group B (Oral Buffer, Positive Control), Metastatic Rates were as Follows:

d. Group B Metastatic Rates:

A. Intestinal: 0%

B. Mesentery: 0%

C. Lymph Node: 27%

D. Lung: 8%

In the Orally Treated, Group B (Oral Buffer, Positive Control), Metastatic Rates were as Follows: In the Topically Treated Group C (Topical Buffer Alone) Metastatic Rates were all Lower:

e. Group C Metastatic Rates:

A. Intestinal: 0%

B. Mesentery: 0%

C. Lymph Node: 22%

D. Lung: 6%

In the Topically Treated Group D (JPM-OEt Alone) Metastatic Rates were all Lower:

a. Group D Metastatic Rates:

A. Intestinal: 22%

B. Mesentery: 12%

C. Lymph Node: 45%

D. Lung: 43%

In the Topically Treated Group E (JPM-OEt Alone) Metastatic Rates were all Lower:

b. Group E Metastatic Rates:

A. Intestinal: 0%

B. Mesentery: 0%

C. Lymph Node: 14%

D. Lung: 3%

There were significant differences observed in survival rates in all treatment groups, Groups B, C, D, and E. As follows:

% of mice surviving to 120 days:

Group A: 20%

Group B: 60%

Group C: 62%

Group D: 38%

Group E: 74%

### Example 14—Topical Buffering Agents of Varying Particle Size to Decrease Primary Tumor Metastases and Increase Survival in Metastatic Breast Cancer

In this experiment, topical applications of buffering agents of various particle size, in formulations of the invention were tested for their ability to synergistically influence the tumor microenvironment to inhibit the spread of metastases and increase overall survival in a mouse model for metastatic breast cancer. The topical formulations of the invention were compared to a “no treatment” control as well as orally delivered buffer as a positive control.

In vivo tests were performed as follows: 72 female Ncr nude mice aged 6 weeks were injected with 5×10⁶ MDA-MB-231/eGFP cells in the mammary fatpad to generate orthotopic “primary” tumors. The following day after tumor inoculation, mice were then randomized into 5 treatment groups as outlined below.

The treatment groups were:

Group A: Untreated Control

Group B: 200 mM sodium bicarbonate drinking water ad libitum

Group C: 50 μL×3 doses daily (total daily dose of 150 μL) of formulation detailed below (Large Particle Size)

Group D: 50 μL×3 doses daily (total daily dose of 150 μL) of formulation detailed below (Medium Particle Size)

Group E: 50 μL×3 doses daily (total daily dose of 150 μL) of formulation detailed below (Small Particle Size)

The formulations of this embodiments of invention are in Table 6 below were as follows:

TABLE 6
Group C (Large Particle Size)

| Ingredient | % Weight |
|---|---|
| Menthol | 0.5% |
| Benzyl Alcohol | 1.0% |
| Cetyl Alcohol | 2.0% |
| Stearic Acid | 2.0% |
| Almond Oil | 3.0% |
| LIP | 14.0% |
| Ethanol | 1.5% |
| Propylene Glycol | 5.0% |
| 30% Pluronic Gel | 18.0% |
| DI Water | 19.0% |
| 50% NaOH Solution | 1.0% |
| Sodium Bicarbonate (100 μm) | 32.0% |
| Durosoft PK-SG | 1.0% |
| Total | 100.0% |

TABLE 7
Group D (Medium Particle Size)

| Ingredient | % Weight |
|---|---|
| Menthol | 0.5% |
| Benzyl Alcohol | 1.0% |
| Cetyl Alcohol | 2.0% |
| Stearic Acid | 2.0% |
| Almond Oil | 3.0% |
| LIP | 14.0% |
| Ethanol | 1.5% |
| Propylene Glycol | 5.0% |
| 30% Pluronic Gel | 18.0% |
| DI Water | 19.0% |
| 50% NaOH Solution | 1.0% |
| Sodium Bicarbonate (65 μm) | 32.0% |
| Durosoft PK-SG | 1.0% |
| Total | 100.0% |

TABLE 8
Group E (Small Particle Size)

| Ingredient | % Weight |
|---|---|
| Menthol | 0.5% |
| Benzyl Alcohol | 1.0% |
| Cetyl Alcohol | 2.0% |
| Stearic Acid | 2.0% |
| Almond Oil | 3.0% |
| LIP | 14.0% |
| Ethanol | 1.5% |
| Propylene Glycol | 5.0% |
| 30% Pluronic Gel | 18.0% |
| DI Water | 19.0% |
| 50% NaOH Solution | 1.0% |
| Sodium Bicarbonate (8.7 μm) | 32.0% |
| Durosoft PK-SG | 1.0% |
| Total | 100.0% |

Application of transdermal agent in treatment groups C, D, and E occurred 3 times/day for 120 days. Volumes of primary tumors in mammary fat pads were measured twice weekly and calculated from orthogonal measurements of external dimensions as (width)²×(length)/2. Surgical resections of primary tumors occurred when tumors reached 350-500 mm³. Mice were euthanized by cervical dislocation when tumor burden became excessive (primary, intraperitoneal, or lymph node >2000 mm³) or when mouse progressed to a moribund state. Survival data were expressed as a Kaplan-Meier curve.

Upon termination of the survival experiment, tumor metastases were identified by gross necropsy. All tumor tissue was fixed in 10% neutral buffered formalin (NBF). The green fluorescent (GFP) tumors were detected using a 470 nm/40 nm excitation filter and imaged using a mounted digital camera. Whole lung images data were analyzed with Adobe Photoshop 5.0 using the “magic wand” tool to select lung area and green fluorescent tumor lesions. Pixel area of the selected images was measured using ImageJ.

There were significant differences observed in metastatic rates in all treatment groups, Groups B, C, D, and E. As follows:

In the Untreated Group a (No Treatment, Negative Control), Metastatic Rates were as Follows:

E. Intestinal: 36%

F. Mesentery: 14%

G. Lymph Node: 64%

H. Lung: 79%

In the Orally Treated, Group B (Oral Buffer, Positive Control), Metastatic Rates were as Follows:

f. Group B Metastatic Rates:

A. Intestinal: 0%

B. Mesentery: 0%

C. Lymph Node: 27%

D. Lung: 8%

In the Topically Treated Group C (Large Particle Size) Metastatic Rates were all Lower:

g. Group C Metastatic Rates:

A. Intestinal: 0%

B. Mesentery: 0%

C. Lymph Node: 22%

D. Lung: 6%

In the Topically Treated Group D (Medium Particle Size) Metastatic Rates were all Lower:

c. Group D Metastatic Rates:

E. Intestinal: 0%

F. Mesentery: 0%

G. Lymph Node: 17%

H. Lung: 5%

In the Topically Treated Group E (Small Particle Size) Metastatic Rates were all Lower:

d. Group E Metastatic Rates:

E. Intestinal: 0%

F. Mesentery: 0%

G. Lymph Node: 5%

H. Lung: 4%

There were significant differences observed in survival rates in all treatment groups, Groups B, C, D, and E. As follows:

% of mice surviving to 120 days:

Group A: 20%

Group B: 60%

Group C: 62%

Group D: 71%

Group E: 80%

### Example 15—Tumor Responsiveness Testing to Topical Buffering Agents and Proteases

In this experiment, tumor biopsy specimens are incubated in various formulations and mediums, including pH neutral mediums and alkaline mediums to determine responsiveness to buffer therapies.

Formulations of the invention are tested in some studies for the ability to modify or reduce protein secretion or in other experiments to inhibit multiple stages of tumor progression with and without coadministration and coformulation of topically applied buffering agents in formulations of the invention.

One measurement in these experiments is to determine if tumor cells are sensitive to particular proteases and by altering their morphology or by acidifying their microenvironment.

Accordingly, in another aspect a diagnostic test is provided for responsiveness of a patient or subject to one or more protease inhibitor as therapeutic agents. Additional diagnostic test provided herein examine responsiveness to one or more protease inhibitor administered in combination with a formulation comprising one or more buffering agent provided herein or formulated with a formulation comprising one or more buffering agent. Proteases inhibitors are administered alone or in combination with formulations comprising one or more buffering agent provided herein to determine if the tumor cells are pH sensitive and therefore may be more responsive if a buffering agent is included in the therapy.

Aspects of the present specification may also be described as follows:

1. A method of treating a proliferative disorder associated with cancer in a patient, the method comprising administering an effective amount of i) one or more protease inhibitor and ii) a formulation for transdermal delivery through the skin of a subject comprising one or more buffering agent to a patient in need thereof, wherein said administration is effective to i) inhibit or prevent the metastasis of tumors or cancer cells, ii) inhibit or prevent the growth of a tumor or tumor cells, iii) inhibit or prevent carcinogenesis, iv) inhibit or prevent the intravasation of tumor cells, or v) improve or extend the duration of remission, or maintain remission of a cancer or tumor.

2. A method according to claim 1, wherein the protease inhibitor is administered transdermally.

3. A method according to claim 1, wherein the protease inhibitor is co-administered with the formulation for transdermal delivery through the skin of a subject comprising one or more buffering agent.

4. A method according to claim 1, wherein the protease inhibitor is formulated with the formulation for transdermal delivery through the skin of a subject comprising one or more buffering agent.

5. A method according to claim 1, wherein the protease inhibitor is administered orally, parenterally or through another rout of administration that is not transdermal.

6. A method according to claim 1, wherein said treating a proliferative disorder inhibits or prevents the metastasis of a tumor or cancer cells.

7. A method according to claim 1, wherein said treating a proliferative disorder inhibits or prevents the growth of tumors or cancer cells.

8. A method according to claim 1, wherein said treating a proliferative disorder inhibits or prevents carcinogenesis.

9. A method according to claim 1, wherein said treating a proliferative disorder inhibits or prevents the intravasation of tumor cells.

10. A method according to claim 1, wherein said treating a proliferative improves or extends the duration of remission or maintains remission of a cancer or tumor.

11. A method of inhibiting or preventing metastasis of tumors, the method comprising administering an effective amount of i) one or more protease inhibitor and ii) a formulation for transdermal delivery through the skin of a subject comprising one or more buffering agent to a patient in need thereof, wherein said administration is effective to inhibit or prevent the metastasis of a tumor or cancer cells.

12. A method of improving, extending the duration of remission, or maintaining remission of a cancer or tumor, the method comprising administering an effective amount of i) one or more protease inhibitor and ii) a formulation for transdermal delivery through the skin of a subject comprising one or more buffering agent to a patient in need thereof, wherein said administration is effective to improve or extend the duration of remission or maintain remission of a cancer or tumor.

13. A method according to claim 1, wherein said formulation for transdermal delivery through the skin of a subject comprises a buffering agent comprising a carbonate salt in an amount between about 10-56% w/w; a penetrant portion in an amount between about 5 to 55% w/w; a detergent portion in an amount of at least 1% w/w; and wherein the formulation comprises water in an amount from 0% w/w up to 70% w/w, and wherein the formulation optionally comprises lecithin in an amount less than about 12% w/w.

14. A method according to claim 1, wherein said formulation for transdermal delivery through the skin of a subject comprises a buffering agent comprising at least one carbonate salt, lysine, tris, a phosphate buffer and/or 2-imidazole-1-yl-3-ethoxycarbonylpropionic acid (IEPA), or a combination thereof in an amount between about 10-56% w/w; and a penetrant portion in an amount between about 44 to 90% w/w, wherein the penetrant portion comprises water in an amount less than about 85% w/w, and wherein the formulation comprises less than about 12% w/w lecithin.

15. A method according to claim 13 or 14, comprising a carbonate salt in an amount between about 7-56% w/w of the formulation.

16. A method according to claim 16, wherein said administration is effective to alter the pH of a tissue or microenvironment proximal to a solid tumor or cancer cells in the patient.

17. A method according to claim 16, wherein the carbonate salt in said formulation is in an amount between about 15-32% w/w of the formulation.

18. A method according to claim 16, wherein the carbonate salt in said formulation is sodium carbonate and/or sodium bicarbonate milled to a particle size is less than 200 μm.

19. A method according to claim 16, wherein a chemotherapeutic or immunotherapeutic agent is co-administered with said formulation.

20. A method according to claim 19, wherein the chemotherapeutic or immunotherapeutic agent is selected from alkylating agents, antibodies and related binding proteins, anthracyclines, antimetabolites, antitumor antibiotics, aromatase inhibitors, taxanes and related compounds, cytoskeletal disruptors, epothilones, histone deacetylace inhibitors, kinase inhibitors, nucleoside analogues, topoisomerase inhibitors, retinoids, and vinca alkaloids and derivatives thereof.

21. A method according to claim 20, wherein the chemotherapeutic or immunotherapeutic agent is an immunotherapeutic agent selected from alemtuzumab, atezolizumab, avelumab, ipilimumab, durvalumab, nivolumab, ofatumumab, rituximab and trastuzumab.

22. A method according to any one of claims 13-15, wherein the penetrant component in said formulation is in an amount between about 18-42% w/w of the formulation.

23. A method according to any one of claims 13-15, wherein the water in said formulation is in an amount between about 15-42% w/w of the formulation.

24. A method according to any one of claims 13-15, wherein the penetrant portion in said formulation comprises an alcohol in an amount less than 5% w/w of the formulation.

25. A method according to any one of claims 13-15, wherein the penetrant portion in said formulation comprises lecithin organogel, an alcohol, a surfactant, and a polar solvent.

26. A method according to any one of claims 13-15, wherein the penetrant portion in said formulation comprises lecithin organogel in an amount less than 5% w/w of the formulation.

27. A method according to claim 22, wherein the lecithin organogel in said formulation is a combination of soy lecithin and isopropyl palmitate.

28. A method according to any one of claims 13-15, wherein the penetrant portion in said formulation comprises lecithin and isopropyl palmitate, undecane, isododecane, isopropyl stearate, or a combination thereof.

29. A method according to any one of claims 13-15, wherein the penetrant portion in said formulation comprises a mixture of xanthan gum, lecithin, sclerotium gum, pullulan, or a combination thereof in an amount less than 5% w/w of the formulation.

30. A method according to any one of claims 13-15, wherein the penetrant portion in said formulation comprises a mixture of caprylic triglycerides and capric triglycerides in amount less than 8% w/w of the formulation.

31. A method according to any one of claims 13-15, wherein the penetrant portion in said formulation comprises phosphatidyl choline in amount less than 12% w/w of the formulation.

32. A method according to any one of claims 13-15, wherein the penetrant portion in said formulation comprises a phospholipid in amount less than 12% w/w of the formulation.

33. A method according to any one of claims 13-15, wherein the penetrant portion in said formulation comprises a mixture of tridecane and undecane in amount less than 5% w/w of the formulation.

34. A method according to any one of claims 13-15, wherein the penetrant portion in said formulation comprises cetyl alcohol in amount less than 5% w/w of the formulation.

35. A method according to any one of claims 13-15, wherein the penetrant portion in said formulation comprises benzyl alcohol in an amount less than about 5 w/w.

36. A method according to any one of claims 13-15 wherein the penetrant portion in said formulation comprises stearic acid in an amount less than 5% w/w of the formulation.

37. A method according to any one of claims 13-15, wherein said formulation comprises a gelling agent in an amount less than 5% w/w of the formulation.

38. A method according to any one of claims 13-15 wherein the detergent portion in said formulation comprises a nonionic surfactant in an amount between about 2-25% w/w of the formulation; and a polar solvent in an amount less than 5% w/w of the formulation.

39. A method according to claim 38, wherein the nonionic surfactant in said formulation is a poloxamer and the polar solvent is water, an alcohol, or a combination thereof.

40. A method according to claim 38, wherein the detergent portion in said formulation comprises poloxamer, propylene glycol, glycerin, ethanol, 50% w/v sodium hydroxide solution, or a combination thereof.

41. A method according to any one of claims 13-15, wherein the detergent portion in said formulation comprises glycerin in an amount less than 3% w/w of the formulation.

42. A method according to any one of claims 13-15, wherein the carbonate salt is sodium carbonate and/or sodium bicarbonate in said formulation is milled to a particle size is less than 70 μm.

43. A method according to any one of claims 13-15, wherein the carbonate salt in said formulation is sodium carbonate and/or sodium bicarbonate milled to a particle size is less than 70 μm, wherein the sodium bicarbonate is solubilized in the formulation in an amount less than 20% w/w of the formulation.

44. A method according to any one of claims 13-15, wherein the carbonate salt in said formulation is sodium carbonate and/or sodium bicarbonate milled to a particle size is less than 70 μm, wherein particle sizes less than about 10 μm have an enhanced penetration thru the skin of a subject.

45. A method according to any one of claims 13-15, wherein said formulation further comprises tranexamic acid in an amount less than 5% w/w of the formulation.

46. A method according to any one of claims 13-15, wherein said formulation further comprises a polar solvent in an amount less than 5% w/w of the formulation.

47. A method according to any one of claims 13-15, wherein said formulation further comprises a humectant, an emulsifier, an emollient, or a combination thereof.

48. A method according to any one of claims 13-15, wherein said formulation further comprises ethylene glycol tetraacetic acid in an amount less than about 5 w/w.

49. A method according to any one of claims 13-15, wherein said formulation further comprises almond oil in an amount less than about 5 w/w.

50. A method according to any one of claims 13-15, wherein said formulation further comprises a mixture of thermoplastic polyurethane and polycarbonate in an amount less than about 5% w/w.

51. A method according to any one of claims 13-15, wherein said formulation further comprises phosphatidylethanolamine in an amount less than about 5 w/w.

52. A method according to any one of claims 13-15, wherein said formulation further comprises an inositol phosphatide in an amount less than about 5 w/w.

53. A method of preventing the intravasation of tumor cells, the method comprising administering, an effective amount of i) one or more protease inhibitor and ii) a formulation for transdermal delivery through the skin of a subject comprising one or more buffering agent to a patient in need thereof, wherein said administration is effective to inhibit or prevent the intravasation of tumor cells.

54. A method of treatment of cancer comprising i) selecting a therapeutic agent comprising. a protease inhibitor, ii) formulating the therapeutic agent in a suitable formulation, iii) administering the formulation comprising the therapeutic agent, and iv) before, during or after step iii), administering a formulation for transdermal delivery comprising one or more buffering agent topically and/or transdermally in an amount effective to i) inhibit or prevent the metastasis of tumors or cancer cells, ii) inhibit or prevent the growth of a tumor or tumor cells, iii) inhibit or prevent carcinogenesis, iv) inhibit or prevent the intravasation of tumor cells, or v) improve or extend the duration of remission, or maintain remission of a cancer or tumor.

55. A method of evaluating a therapeutic agent or formulation for the treatment for cancer, the method comprising i) administering one or more protease inhibitor and ii) administering a formulation for transdermal delivery through the skin of a subject comprising one or more buffering agent, wherein said administration is evaluated for effectiveness to i) inhibit or prevent the metastasis of tumors or cancer cells, ii) inhibit or prevent the growth of a tumor or tumor cells, iii) inhibit or prevent carcinogenesis, iv) inhibit or prevent the intravasation of tumor cells, or v) improve or extend the duration of remission, or maintain remission of a cancer or tumor, wherein step i) and be performed before, after, or concurrent with step ii).

56. A method according to any one of the preceding claims wherein said formulation for transdermal delivery comprises a buffering agent comprising a carbonate salt in an amount between about 10-45% w/w; a penetrant portion in an amount between about 5 to 55% w/w; a detergent portion in an amount between about 1 to 15% w/w; and wherein the formulation comprises water in an amount between about 15 to 65% w/w, and wherein the formulation comprises less than about 12% w/w lecithin.

57. A method according to claim 56, wherein the administering is performed topically by directly contacting the skin of said subject with the formulation provided to said subject.

58. A method according to claim 57, wherein prior to application of the formulation skin of said patient is pretreated by abrasion, tape-stripping, microderm-abrasion, or microneedling

59. A medical formulation kit, the kit comprising a lotion for administering topically and/or transdermally a formulation comprising a buffering agent and administration directions that includes instructions for amounts and use for a medical professional.

60. A method according to claim 56, wherein the carbonate salt in said formulation is in an amount between about 7-32% w/w of the formulation.

61. A method according to claim 60, wherein the carbonate salt in said formulation is in an amount between about 15-32% w/w of the formulation.

62. A method according to claim 60, wherein the penetrant component in said formulation is in an amount between about 18-42% w/w of the formulation.

63. A method according to claim 60, wherein the water in said formulation is in an amount between about 15-42% w/w of the formulation.

64. A method according to claim 60, wherein the penetrant portion in said formulation comprises an alcohol in an amount less than 5% w/w of the formulation.

65. A method according to claim 60, wherein the penetrant portion in said formulation comprises lecithin organogel, an alcohol, a surfactant, and a polar solvent.

66. A method according to claim 60, wherein the penetrant portion in said formulation comprises lecithin organogel in an amount less than 5% w/w of the formulation.

67. A method according to claim 18, wherein the lecithin organogel in said formulation is a combination of soy lecithin and isopropyl palmitate.

68. A method according to claim 60, wherein the penetrant portion in said formulation comprises lecithin and isopropyl palmitate, undecane, isododecane, isopropyl stearate, or a combination thereof.

69. A method according to claim 60, wherein the penetrant portion in said formulation comprises a mixture of xanthan gum, lecithin, sclerotium gum, pullulan, or a combination thereof in an amount less than 5% w/w of the formulation.

70. A method according to claim 60, wherein the penetrant portion in said formulation comprises a mixture of caprylic triglycerides and capric triglycerides in amount less than 8% w/w of the formulation.

71. A method according to claim 60, wherein the penetrant portion in said formulation comprises phosphatidyl choline in amount less than 12% w/w of the formulation.

72. A method according to claim 60, wherein the penetrant portion in said formulation comprises a phospholipid in amount less than 12% w/w of the formulation.

73. A method according to claim 60, wherein the penetrant portion in said formulation comprises a mixture of tridecane and undecane in amount less than 5% w/w of the formulation.

74. A method according to claim 60, wherein the penetrant portion in said formulation comprises cetyl alcohol in amount less than 5% w/w of the formulation.

75. A method according to claim 60, wherein the penetrant portion in said formulation comprises benzyl alcohol in an amount less than about 5 w/w.

76. A method according to claim 60 wherein the penetrant portion in said formulation comprises stearic acid in an amount less than 5% w/w of the formulation.

77. A method according to claim 60, wherein said formulation comprises a gelling agent in an amount less than 5% w/w of the formulation.

78. A method according to claim 60 wherein the detergent portion in said formulation comprises a nonionic surfactant in an amount between about 2-25% w/w of the formulation; and a polar solvent in an amount less than 5% w/w of the formulation.

79. A method according to claim 81, wherein the nonionic surfactant in said formulation is a poloxamer and the polar solvent is water, an alcohol, or a combination thereof.

80. A method according to claim 81, wherein the detergent portion in said formulation comprises poloxamer, propylene glycol, glycerin, ethanol, 50% w/v sodium hydroxide solution, or a combination thereof.

81. A method according to claim 60, wherein the detergent portion in said formulation comprises glycerin in an amount less than 3% w/w of the formulation.

82. A method according to claim 60, wherein the carbonate salt in said formulation is sodium carbonate and/or sodium bicarbonate milled to a particle size is less than 200 μm.

83. A method according to claim 60, wherein the carbonate salt is sodium carbonate and/or sodium bicarbonate in said formulation is milled to a particle size is less than 70 μm.

84. A method according to claim 60, wherein the carbonate salt in said formulation is sodium carbonate and/or sodium bicarbonate milled to a particle size is less than 70 μm, wherein the sodium bicarbonate is solubilized in the formulation in an amount less than 20% w/w of the formulation.

85. A method according to claim 60, wherein the carbonate salt in said formulation is sodium carbonate and/or sodium bicarbonate milled to a particle size is less than 70 μm, wherein particle sizes less than about 10 μm have an enhanced penetration thru the skin of a subject.

86. A method according to claim 60, wherein said formulation further comprises tranexamic acid in an amount less than 5% w/w of the formulation.

87. A method according to claim 60, wherein said formulation further comprises a polar solvent in an amount less than 5% w/w of the formulation.

88. A method according to claim 60, wherein said formulation further comprises a humectant, an emulsifier, an emollient, or a combination thereof.

89. A method according to claim 60, wherein said formulation further comprises ethylene glycol tetraacetic acid in an amount less than about 5 w/w.

90. A method according to claim 60, wherein said formulation further comprises almond oil in an amount less than about 5 w/w.

91. A method according to claim 60, wherein said formulation further comprises a mixture of thermoplastic polyurethane and polycarbonate in an amount less than about 5 w/w.

92. A method according to claim 60, wherein said formulation further comprises phosphatidylethanolamine in an amount less than about 5 w/w.

93. A method according to claim 60, wherein said formulation further comprises an inositol phosphatide in an amount less than about 5 w/w.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating a proliferative disorder associated with cancer in a patient, the method comprising administering an effective amount of i) one or more protease inhibitor and ii) a formulation for transdermal delivery through the skin of a subject comprising one or more buffering agent to a patient in need thereof, wherein said administration is effective to i) inhibit or prevent the metastasis of tumors or cancer cells, ii) inhibit or prevent the growth of a tumor or tumor cells, iii) inhibit or prevent carcinogenesis, iv) inhibit or prevent the intravasation of tumor cells, or v) improve or extend the duration of remission, or maintain remission of a cancer or tumor.

2. A method according to claim 1, wherein the protease inhibitor is administered transdermally.

3. A method according to claim 1, wherein the protease inhibitor is co-administered with the formulation for transdermal delivery through the skin of a subject comprising one or more buffering agent.

4. A method according to claim 1, wherein the protease inhibitor is formulated with the formulation for transdermal delivery through the skin of a subject comprising one or more buffering agent.

5. A method according to claim 1, wherein the protease inhibitor is administered orally, parenterally or through another rout of administration that is not transdermal.

6. A method according to claim 1, wherein said treating a proliferative disorder inhibits or prevents the metastasis of a tumor or cancer cells.

7. A method according to claim 1, wherein said treating a proliferative disorder inhibits or prevents the growth of tumors or cancer cells.

8. A method according to claim 1, wherein said treating a proliferative disorder inhibits or prevents carcinogenesis.

9. A method according to claim 1, wherein said treating a proliferative disorder inhibits or prevents the intravasation of tumor cells.

10. A method according to claim 1, wherein said treating a proliferative improves or extends the duration of remission or maintains remission of a cancer or tumor.

11. A method of inhibiting or preventing metastasis of tumors, the method comprising administering an effective amount of i) one or more protease inhibitor and ii) a formulation for transdermal delivery through the skin of a subject comprising one or more buffering agent to a patient in need thereof, wherein said administration is effective to inhibit or prevent the metastasis of a tumor or cancer cells.

12. A method of improving, extending the duration of remission, or maintaining remission of a cancer or tumor, the method comprising administering an effective amount of i) one or more protease inhibitor and ii) a formulation for transdermal delivery through the skin of a subject comprising one or more buffering agent to a patient in need thereof, wherein said administration is effective to improve or extend the duration of remission or maintain remission of a cancer or tumor.

13. A method according to claim 1, wherein said formulation for transdermal delivery through the skin of a subject comprises a buffering agent comprising a carbonate salt in an amount between about 10-56% w/w; a penetrant portion in an amount between about 5 to 55% w/w; a detergent portion in an amount of at least 1% w/w; and wherein the formulation comprises water in an amount from 0% w/w up to 70% w/w, and wherein the formulation optionally comprises lecithin in an amount less than about 12% w/w.

14. A method according to claim 1, wherein said formulation for transdermal delivery through the skin of a subject comprises a buffering agent comprising at least one carbonate salt, lysine, tris, a phosphate buffer and/or 2-imidazole-1-yl-3-ethoxycarbonylpropionic acid (IEPA), or a combination thereof in an amount between about 10-56% w/w; and a penetrant portion in an amount between about 44 to 90% w/w, wherein the penetrant portion comprises water in an amount less than about 85% w/w, and wherein the formulation comprises less than about 12% w/w lecithin.

15. A method according to claim 13, comprising a carbonate salt in an amount between about 7-56% w/w of the formulation.

16. A method according to claim 16, wherein said administration is effective to alter the pH of a tissue or microenvironment proximal to a solid tumor or cancer cells in the patient.

17. A method according to claim 16, wherein the carbonate salt in said formulation is in an amount between about 15-32% w/w of the formulation.

18. A method according to claim 16, wherein the carbonate salt in said formulation is sodium carbonate and/or sodium bicarbonate milled to a particle size is less than 200 μm.

19. A method according to claim 16, wherein a chemotherapeutic or immunotherapeutic agent is co-administered with said formulation.

20. A method according to claim 19, wherein the chemotherapeutic or immunotherapeutic agent is selected from alkylating agents, antibodies and related binding proteins, anthracyclines, antimetabolites, antitumor antibiotics, aromatase inhibitors, taxanes and related compounds, cytoskeletal disruptors, epothilones, histone deacetylace inhibitors, kinase inhibitors, nucleoside analogues, topoisomerase inhibitors, retinoids, and vinca alkaloids and derivatives thereof.
